(19) United States
(12) Patent Application Publication
(10) Pub. No.: US 2022/0333140 A1
(43) Pub. Date: Oct. 20, 2022

(54) PROCESS FOR IMPROVING CARBON CONVERSION EFFICIENCY

(71) Applicant: LanzaTech, Inc., Skokie, IL (US)

(72) Inventors: Robert John Conrado, Washington, DC (US); Sean Dennis Simpson, Evanston, IL (US); Michael Emerson Martin, Chicago, IL (US); Jason Carl Bromley, Chicago, IL (US); Richard Russell Rosin, Glencoe, IL (US)

(21) Appl. No.: 17/658,585

(22) Filed: Apr. 8, 2022

Related U.S. Application Data
(60) Provisional application No. 63/173,247, filed on Apr. 9, 2021.

(51) Int. Cl. C12P 7/06; C01B 32/40; B01J 19/24; C10L 1/02; C12M 1/00; C25B 1/04; C25B 15/08

(52) U.S. Cl. CPC C12P 7/06; C01B 32/40; B01J 19/2465; C10L 1/02; C12M 21/12; C25B 1/04; C25B 15/081; C10L 2290/26; C12M 43/04

(57) ABSTRACT

The disclosure provides for the integration of a CO-consuming process, such as a gas fermentation process, with a CO₂ to CO conversion system. The disclosure is capable of utilizing a CO₂-comprising gaseous substrate generated by an industrial process and provides for one or more removal modules to remove at least one constituent from a CO₂-comprising gaseous substrate prior to passage of the gaseous substrate to a CO₂ to CO conversion system. The disclosure may further comprise one or more pressure modules, one or more CO₂ concentration modules, one or more O₂ separation modules, and/or a water electrolysis module. Carbon conversion efficiency is increased by recycling CO₂ produced by a CO-consuming process to the CO₂ to CO conversion process.

### CROSS REFERENCE TO RELATED APPLICATIONS

This application claims the benefit of U.S. Provisional Patent Application No. 63/173,247, filed Apr. 9, 2021, the entirety of which is incorporated herein by reference.

### FIELD

The disclosure relates to processes and methods for improving carbon conversion efficiency. In particular, the disclosure relates to the combination of a carbon monoxide-consuming process with an industrial process or with syngas, wherein gas from the industrial process or syngas undergoes treatment and conversion, and carbon dioxide produced by the carbon monoxide-consuming process is recycled to increase product yield.

### BACKGROUND

Carbon dioxide (CO₂) accounts for about 76% of global greenhouse gas emissions from human activities, with methane (16%), nitrous oxide (6%), and fluorinated gases (2%) accounting for the balance (United States Environmental Protection Agency). Reduction of greenhouse gas emissions, particularly CO₂, is critical to halt the progression of global warming and the accompanying shifts in climate and weather.

It has long been recognized that catalytic processes, such as the Fischer-Tropsch process, may be used to convert gases comprising CO₂, carbon monoxide (CO), and/or hydrogen (H₂) into a variety of fuels and chemicals. Recently, however, gas fermentation has emerged as an alternative platform for the biological fixation of such gases. In particular, C1-fixing microorganisms have been demonstrated to convert gases comprising CO₂, CO, CH₄, and/or H₂ into products such as ethanol and 2,3-butanediol.

Such gases may be derived, for example, from industrial processes, including gas emissions from carbohydrate fermentation, gas fermentation, cement making, pulp and paper making, steel making, oil refining and associated processes, petrochemical production, coke production, anaerobic or aerobic digestion, gasification, natural gas extraction, oil extraction, metallurgical processes, production and/or refinement of aluminum, copper, and/or ferroalloys, geological reservoirs, Fischer-Tropsch processes, methanol production, pyrolysis, steam methane reforming, dry methane reforming, partial oxidation of biogas or natural gas, and autothermal reforming of biogas or natural gas.

To optimize the usage of these gases in CO-consuming processes, such as C1-fixing fermentation processes, an industrial gas may require a combination of treatment and conversion. Accordingly, there remains a need for improved integration of industrial processes with CO-consuming processes, including processes for treatment and conversion of industrial gases, thereby optimizing carbon conversion efficiency.

### BRIEF SUMMARY

A process for improving carbon conversion efficiency is disclosed. The process comprises a) passing a CO₂-containing gaseous substrate from an industrial process, a synthesis gas process, or a combination thereof, to at least one removal module for removal of at least one constituent from the CO₂-containing gaseous substrate, to produce a treated gas stream, comprising at least a portion of CO₂; b) passing the treated gas stream to a CO₂ to CO conversion system for conversion of at least a portion of the CO₂ to produce a first CO-enriched stream, wherein the CO₂ to CO conversion system is selected from reverse water gas reaction system, thermo-catalytic conversion system, electro-catalytic conversion system, partial combustion system, or plasma conversion system; c) passing at least a portion of the first CO-enriched stream to a bioreactor comprising a culture of at least one C1-fixing microorganism; d) fermenting the culture to produce one or more fermentation products and a post-fermentation gaseous substrate comprising CO₂ and H₂; e) passing at least a portion of the post-fermentation gaseous substrate comprising CO₂ and H₂ to at least one removal module for removal of at least one constituent from the post-fermentation gaseous substrate to produce a treated gas stream; and f) recycling at least a portion of the treated stream to the CO₂ to CO conversion system.

The industrial process may be selected from industrial process is selected from fermentation, carbohydrate fermentation, sugar fermentation, cellulosic fermentation, gas fermentation, cement making, pulp and paper making, steel making, oil refining, petrochemical production, coke production, anaerobic digestion, aerobic digestion, natural gas extraction, oil extraction, geological reservoirs, metallurgical processes, refinement of aluminium, copper and or ferroalloys, for production of aluminium, copper, and or ferroalloys, direct air capture, or any combination thereof; or the synthesis gas process is selected from gasification of coal, gasification of refinery residues, gasification of petroleum coke, gasification of biomass, gasification of lignocellulosic material, gasification of waste wood, gasification of black liquor, gasification of municipal solid waste, gasification of municipal liquid waste, gasification of industrial solid waste, gasification of industrial liquid waste, gasification of refuse derived fuel, gasification of sewerage, gasification of sewerage sludge, gasification of sludge from wastewater treatment, gasification of biogas, reforming of landfill gas, reforming of biogas, reforming of methane, naphtha reforming, partial oxidation, or any combination thereof.

The H₂-rich stream may be generated using a water electrolyzer and at least a portion of the H₂-rich stream may be blended with the CO-enriched stream prior to being passed to the bioreactor or at least a portion of the H₂-rich stream may be passed to the bioreactor; or both at least a portion of the H₂-rich stream may be blended with the CO-enriched stream prior to being passed to the bioreactor and at least a portion of the H₂-rich stream may be passed to the bioreactor.

The process the CO-enriched stream from the CO₂ to CO conversion system may be passed to a removal module prior to being passed to the bioreactor. The at least one constituent may be removed from a) the CO-enriched stream; b) the CO₂-containing gas substrate; and or c) the post-fermentation gaseous substrate; and may be selected from sulfur-comprising compounds, aromatic compounds, alkynes, alkenes, alkanes, olefins, nitrogen-comprising compounds, oxygen, phosphorous-comprising compounds, particulate matter, solids, oxygen, halogenated compounds, silicon-comprising compounds, carbonyls, metals, alcohols, esters, ketones, peroxides, aldehydes, ethers, tars, and naphthalene. The at least one constituent removed from the CO-enriched stream by the removal module may comprise oxygen. The at least one constituent removed and/or converted may be a microbe inhibitor and/or a catalyst inhibitor. The at least one constituent removed may be produced, introduced, and/or concentrated by the fermentation step. The at least one constituent removed may be produced, introduced, and/or concentrated by the CO₂ to CO conversion system.

The C1-fixing microorganism may be a carboxydotrophic bacterium. The carboxydotrophic bacterium may be selected from the group comprising Moorella, Clostridium, Ruminococcus, Acetobacterium, Eubacterium, Butyribacterium, Oxobacter, Methanosarcina, and Desulfotomaculum. The carboxydotrophic bacterium may be Clostridium autoethanogenum.

The CO₂-containing gaseous substrate may be passed to a carbon dioxide concentration module to enhance the level of carbon dioxide contained in (i) the CO₂-containing gaseous substrate prior to the CO₂-containing gaseous substrate being passed to the one or more removal module, (ii) the treated gas stream comprising at least a portion of carbon dioxide prior to the treated gas stream being passed to the water electrolyzer; and/or (iii) the post-fermentation gaseous substrate prior to the post-fermentation gaseous substrate being passed to the one or more removal modules, or the bioreactor. The CO₂-containing gaseous substrate from the industrial process, the synthesis gas process, or the combination thereof may be passed to a pressure module to produce a pressurized CO₂-containing gas stream and then passing the pressurized CO₂-containing gas stream to the first removal module. The CO-enriched stream may be passed to a pressure module to produce a pressurized CO-stream and the pressurized CO-stream may be passed to the bioreactor.

The at least one removal module may be selected from hydrolysis module, acid gas removal module, deoxygenation module, catalytic hydrogenation module, particulate removal module, chloride removal module, tar removal module, or hydrogen cyanide polishing module.

The at least one fermentation product may be selected from ethanol, butyrate, 2,3-butanediol, lactate, butene, butadiene, methyl ethyl ketone, ethylene, acetone, isopropanol, lipids, 3-hydroypropionate, terpenes, fatty acids, 2-butanol, 1,2-propanediol, or 1-propanol. The at least one of the fermentation product maybe further converted to at least one component of diesel, jet fuel, and/or gasoline. The at least one fermentation product may comprise microbial biomass. At least a portion of the microbial biomass may be processed to produce at least a portion of animal feed.

The CO-enriched stream may comprise at least a portion of oxygen, and at least a portion of the CO-enriched stream may be passed to an oxygen separation module to separate at least a portion of oxygen from the carbon monoxide enriched stream.

A process for improving process economics of an integrated industrial fermentation system is also disclosed. The process comprises a) passing a feedstock comprising water to a water electrolyzer, wherein at least a portion of the water is converted to H₂ and O₂; b) passing a CO₂-containing gaseous substrate to a reverse water gas shift process to generate a CO-enriched stream; c) passing at least a portion of the H₂ and at least a portion of the CO-enriched stream from the reverse water gas shift process to a bioreactor containing a culture of at least one C1-fixing microorganism; d) fermenting the culture to produce one or more fermentation products and a post-fermentation gaseous substrate comprising CO₂ and H₂; and e) passing at least a portion of the post-fermentation gaseous substrate back to the reverse water gas shift process. The amount of CO₂ in the post-fermentation gaseous substrate exiting the bioreactor may be greater than an amount of unconverted CO₂ introduced to the bioreactor. The fermentation process may perform the function of a CO₂ concentration module.

### BRIEF DESCRIPTION OF THE DRAWINGS

FIG. 1A shows a process integration scheme depicting integration of a removal module, a CO₂ to CO conversion system, and an optional water electrolysis module with a CO-consuming process. FIG. 1B further shows a pressure module prior to a removal module.

FIG. 1C further shows a pressure module prior to a CO-consuming process.

FIG. 2 shows a process integration scheme depicting integration of a removal module, a CO₂ to CO conversion system, an optional O₂ separation module, and an optional water electrolysis module with a CO-consuming process.

FIG. 3 shows a process integration scheme depicting integration of an optional CO₂ concentration module prior to a removal module, a CO₂ to CO conversion system, an optional water electrolysis module, and an optional O₂ separation module with a CO-consuming process.

FIG. 4 shows a process integration scheme depicting integration of an optional CO₂ concentration module following a removal module, a CO₂ to CO conversion system, an optional water electrolysis module, and an optional O₂ separation module with a CO-consuming process.

FIG. 5 shows a process integration scheme depicting integration of a water electrolysis module following an optional pressure module, wherein a portion of the gas from the water electrolysis module is blended with the gas from the CO₂ to CO conversion system prior to being passed to the CO-consuming process.

FIG. 6 shows a process integration scheme depicting integration of a further removal module following a CO₂ to CO conversion system.

### DETAILED DESCRIPTION

The inventors have identified that the integration of a CO₂-generating industrial process with a CO-consuming process, as well as a removal process prior to a CO₂ to CO conversion process, is capable of providing substantial benefits to the CO₂-generating industrial process and the CO-consuming process, which may be a C1-fixing fermentation process.

The term “industrial process” refers to a process for producing, converting, refining, reforming, extracting, or oxidizing a substance involving chemical, physical, electrical, and/or mechanical steps. Exemplary industrial processes include, but are not limited to, carbohydrate fermentation, gas fermentation, cement making, pulp and paper making, steel making, oil refining and associated processes, petrochemical production, coke production, anaerobic or aerobic digestion, gasification (such as gasification of biomass, liquid waste streams, solid waste streams, municipal streams, fossil resources including natural gas, coal and oil), natural gas extraction, oil extraction, metallurgical processes, production and/or refinement of aluminum, copper, and/or ferroalloys, geological reservoirs, Fischer-Tropsch processes, methanol production, pyrolysis, steam methane reforming, dry methane reforming, partial oxidation of biogas or natural gas, direct air capture, and autothermal reforming of biogas or natural gas. In these embodiments, the substrate and/or C1-carbon source may be captured from the industrial process before it is emitted into the atmosphere, using any convenient method.

The terms “gas from an industrial process,” “gas source from an industrial process,” and “gaseous substrate from an industrial process” can be used interchangeably to refer to an off-gas from an industrial process, a by-product of an industrial process, a co-product of an industrial process, a gas recycled within an industrial process, and/or a gas used within an industrial facility for energy recovery. In some embodiments, a gas from an industrial process is a pressure swing adsorption (PSA) tail gas. In some embodiments, a gas from an industrial process is a gas obtained through a CO₂ extraction process, which may involve amine scrubbing or use of a carbonic anhydrase solution.

“C1” refers to a one-carbon molecule, for example, CO, CO₂, methane (CH₄), or methanol (CH₃OH). “C1-oxygenate” refers to a one-carbon molecule that also comprises at least one oxygen atom, for example, CO, CO₂, or CH₃OH. “C1-carbon source” refers a one carbon-molecule that serves as a partial or sole carbon source for a microorganism of the disclosure. For example, a C1-carbon source may comprise one or more of CO, CO₂, CH₄, CH₃OH, or formic acid (CH₂O₂). Preferably, a C1-carbon source comprises one or both of CO and CO₂. A “C1-fixing microorganism” is a microorganism that has the ability to produce one or more products from a C1-carbon source. Typically, a microorganism of the disclosure is a C1-fixing bacterium.

“Substrate” refers to a carbon and/or energy source. Typically, the substrate is gaseous and comprises a C1-carbon source, for example, CO, CO₂, and/or CH₄. Preferably, the substrate comprises a C1-carbon source of CO or CO and CO₂. The substrate may further comprise other non-carbon components, such as H₂, N₂, or electrons. As used herein, “substrate” may refer to a carbon and/or energy source for a microorganism of the disclosure.

The term “co-substrate” refers to a substance that, while not necessarily being the primary energy and material source for product synthesis, can be utilized for product synthesis when combined with another substrate, such as the primary substrate.

A “CO₂-comprising gaseous substrate,” “CO₂-comprising gas,” or “CO₂-comprising gaseous source” may include any gas that comprises CO₂. The gaseous substrate will typically comprise a significant proportion of CO₂, preferably at least about 5% to about 100% CO₂ by volume. Additionally, the gaseous substrate may comprise one or more of hydrogen (H₂), oxygen (O₂), nitrogen (N₂), and/or CH₄. As used herein, CO, H₂, and CH₄ may be referred to as “energy-rich gases.”

The term “carbon capture” as used herein refers to the sequestration of carbon compounds including CO₂ and/or CO from a stream comprising CO₂ and/or CO and either a) converting the CO₂ and/or CO into products, b) converting the CO₂ and/or CO into substances suitable for long term storage, c) trapping the CO₂ and/or CO in substances suitable for long term storage, or d) a combination of these processes.

The terms “increasing the efficiency,” “increased efficiency,” and the like refer to an increase in the rate and/or output of a reaction, such as an increased rate of converting the CO₂ and/or CO into products and/or an increased product concentration. When used in relation to a fermentation process, “increasing the efficiency” includes, but is not limited to, increasing one or more of the rate of growth of microorganisms catalyzing a fermentation, the growth and/or product production rate at elevated product concentrations, the volume of desired product produced per volume of substrate consumed, the rate of production or level of production of the desired product, and the relative proportion of the desired product produced compared with other by-products of the fermentation.

“Reactant” as used herein refers to a substance that is present in a chemical reaction and is consumed during the reaction to produce a product. A reactant is a starting material that undergoes a change during a chemical reaction. In particular embodiments, a reactant includes, but is not limited to, CO and/or H₂. In particular embodiments, a reactant is CO₂.

A “CO-consuming process” refers to a process wherein CO is a reactant; CO is consumed to produce a product. A non-limiting example of a CO-consuming process is a C1-fixing gas fermentation process. A CO-consuming process may involve a CO₂-producing reaction. For example, a CO-consuming process may result in the production of at least one product, such as a fermentation product, as well as CO₂. In another example, acetic acid production is a CO-consuming process, wherein CO is reacted with methanol under pressure.

“Gas stream” refers to any stream of substrate which is capable of being passed, for example, from one module to another, from one module to a CO-consuming process, and/or from one module to a carbon capture means.

Gas streams typically will not be a pure CO₂ stream and will comprise proportions of at least one other component. For instance, each source may have differing proportions of CO₂, CO, H₂, and various constituents. Due to the varying proportions, a gas stream must be processed prior to being introduced to a CO-consuming process. Processing of the gas stream includes the removal and/or conversion of various constituents that may be microbe inhibitors and/or catalyst inhibitors. Preferably, catalyst inhibitors are removed and/or converted prior to being passed to the CO₂ to CO conversion process, and microbe inhibitors are removed and/or converted prior to being passed to a CO-consuming process. Additionally, a gas stream may need to undergo one or more concentration steps whereby the concentration of CO and/or CO₂ is increased. Preferably, a gas stream will undergo a concentration step to increase the concentration of CO₂ prior to being passed to the CO₂ to CO conversion process. It has been found that higher concentrations of CO₂ being passing into the CO₂ to CO conversion process results in higher concentrations of CO coming out of the CO₂ to CO conversion process.

“Removal module,” “contaminant removal module,” “clean-up module,” “processing module,” and the like include technologies that are capable of either converting and/or removing at least one constituent from a gas stream. Non-limiting examples of removal modules include hydrolysis modules, acid gas removal modules, deoxygenation modules, catalytic hydrogenation modules, particulate removal modules, chloride removal modules, tar removal modules, and hydrogen cyanide polishing modules.

The terms “constituents,” “contaminants,” and the like, as used herein, refer to the microbe inhibitors and/or catalyst inhibitors that may be found in a gas stream. In particular embodiments, the constituents include, but are not limited to, sulfur-comprising compounds, aromatic compounds, alkynes, alkenes, alkanes, olefins, nitrogen-comprising compounds, phosphorous-comprising compounds, particulate matter, solids, oxygen, halogenated compounds, silicon-comprising compounds, carbonyls, metals, alcohols, esters, ketones, peroxides, aldehydes, ethers, tars, and naphthalene. Preferably, the constituent removed by the removal module does not include CO₂.

“Microbe inhibitors” as used herein refer to one or more constituents that slow down or prevent a particular chemical reaction or other process, including the microbe. In particular embodiments, the microbe inhibitors include, but are not limited to, oxygen (O₂), hydrogen cyanide (HCN), acetylene (C₂H₂), and BTEX (benzene, toluene, ethyl benzene, xylene).

“Catalyst inhibitor,” “adsorbent inhibitor,” and the like, as used herein, refer to one or more substances that decrease the rate of or prevent a chemical reaction. In particular embodiments, the catalyst inhibitors may include, but are not limited to, hydrogen sulfide (H₂S) and carbonyl sulfide (COS).

In certain instances, at least one constituent removed is produced, introduced, and/or concentrated by a fermentation step. One or more of these constituents may be present in a post-fermentation gaseous substrate. For example, sulfur, in the form of H₂S may be produced, introduced, and/or concentrated by a fermentation step. In particular embodiments, hydrogen sulfide is introduced in the fermentation step. In various embodiments, the post-fermentation gaseous substrate comprises at least a portion of hydrogen sulfide. Hydrogen sulfide may be a catalyst inhibitor. Hydrogen sulfide may be inhibiting to particular the CO₂ to CO conversion process, if employed. In order to pass a non-inhibiting post-fermentation gaseous substrate to a CO₂ to CO conversion process, at least a portion of the hydrogen sulfide, or other constituent present in the post-fermentation gaseous substrate, may need to be removed by one or more removal module. In another embodiment, acetone may be produced by a fermentation step, and charcoal may be used as a removal module.

The terms “treated gas” and “treated gas stream” refer to a gas stream that has been passed through at least one removal module and has had one or more constituent removed and/or converted. For example, a “CO₂-treated gas stream” refers to a CO₂-comprising gas that has passed through one or more removal module.

“Concentration module” and the like refer to technology capable of increasing the level of a particular component in a gas stream. In particular embodiments, the concentration module is a CO₂ concentration module, wherein the proportion of CO₂ in the gas stream leaving the CO₂ concentration module is higher relative to the proportion of CO₂ in the gas stream prior to being passed to the CO₂ concentration module. In some embodiments, a CO₂ concentration module uses deoxygenation technology to remove O₂ from a gas stream and thus increase the proportion of CO₂ in the gas stream. In some embodiments, a CO₂ concentration module uses pressure swing adsorption (PSA) technology to remove H₂ from a gas stream and thus increase the proportion of CO₂ in the gas stream. In certain instances, a fermentation process performs the function of a CO₂ concentration module. In some embodiments, a gas stream from a concentration module is passed to a carbon capture and sequestration (CCS) unit or an enhanced oil recovery (EOR) unit.

The term “CO₂ to CO conversion system” as used herein refers to at least one unit selected from reverse water gas reaction system, thermo-catalytic conversion system, electro-catalytic conversion system, partial combustion system and plasma conversion system. Previously, a CO₂ electrolysis module was employed as a process to convert at least some collected CO₂ to CO. However, in some applications electricity may be cost prohibitive, not sustainable, not reliable, or not easily available. Therefore, a need exists for another solution to utilize available CO₂ waste gas. The CO₂ to CO conversion system provides such solution. A particular embodiment the CO₂ to CO conversion system is a reverse water gas reaction unit or system.

The term “reverse water gas reaction unit”/“rWGR unit” as used herein refers to a unit or system used for producing water from carbon dioxide and hydrogen, with carbon monoxide as a side product. The term “water gas” is defined as a fuel gas consisting mainly of carbon monoxide (CO) and hydrogen (H₂). The term ‘shift’ in water-gas shift means changing the water gas composition (CO:H₂) ratio. The ratio can be increased by adding CO₂ or reduced by adding steam to the reactor. The reverse water gas reaction unit may comprise a single stage or more than one stage. The different stages may be conducted at different temperatures and may use different catalysts.

The term “thermo-catalytic conversion”, another suitable CO₂ to CO conversion system, refers to a process to disrupt the stable atomic and molecular bonds of CO₂ and other reactants over a catalyst by using thermal energy as the driving force of the reaction to produce CO. Since CO₂ molecules are thermodynamically and chemically stable, if CO₂ is used as a single reactant, large amounts of energy are required. Therefore, often other substances such as hydrogen are used as a co-reactant to make the thermodynamic process easier. Many catalysts are known for the process such as metals and metal oxides as well as nano-sized catalyst metal-organic frameworks. Various carbon materials have been employed as carriers for the catalysts.

The term “partial combustion system” as used herein refers to a system where oxygen supplies at least a portion of the oxidant requirement for partial oxidation and the reactants carbon dioxide and water present therein are substantially converted to carbon monoxide and hydrogen.

The term “plasma conversion” refers to CO₂ conversion process, focusing on the combination of plasma with catalysts, called as plasma-catalysis. “Plasma” also called the “fourth state of matter,” is an ionized gas consisting of electrons, various types of ions, radicals, excited atoms, and molecules, besides neutral ground state molecules. The three most common plasma types for CO₂ conversion are: dielectric barrier discharges (DBDs), microwave (MW) plasmas, and gliding arc (GA) plasmas.

“Plasma conversion system” for CO₂ conversion comprises (i) high process versatility, allowing different kinds of reactions to be carried out (e.g., pure CO₂ splitting, as well as CO₂ conversion in the presence of a H-source, such as CH₄, H₂ or H₂O); (ii) low investment and operating costs; (iii) does not require the use of rare earth metals; (iv) a very modular setting, as plasma reactors scale up linearly with the plant output, allowing on-demand production; and (v) it can be very easily combined with (various kinds of) renewable electricity.

The terms “electrolysis module” and “electrolyzer” can be used interchangeably to refer to a unit that uses electricity to drive a non-spontaneous reaction. Electrolysis technologies are known in the art. Exemplary processes include alkaline water electrolysis, proton, or anion exchange membrane (PEM, AEM) electrolysis, and solid oxide electrolysis (SOE) (Ursua et al., Proceedings of the IEEE 100(2):410-426, 2012; Jhong et al., Current Opinion in Chemical Engineering 2:191-199, 2013). The term “faradaic efficiency” is a value that references the number of electrons flowing through an electrolyzer and being transferred to a reduced product rather than to an unrelated process. SOE modules operate at elevated temperatures. Below the thermoneutral voltage of an electrolysis module, an electrolysis reaction is endothermic. Above the thermoneutral voltage of an electrolysis module, an electrolysis reaction is exothermic. In some embodiments, an electrolysis module is operated without added pressure. In some embodiments, an electrolysis module is operated at a pressure of 5-10 bar.

A “CO₂ electrolysis module” refers to a unit capable of splitting CO₂ into CO and O₂ and is defined by the following stoichiometric reaction: 2CO₂+electricity→2CO+O₂. The use of different catalysts for CO₂ reduction impact the end product. Catalysts including, but not limited to, Au, Ag, Zn, Pd, and Ga catalysts, have been shown effective to produce CO from CO₂. In some embodiments, the pressure of a gas stream leaving a CO₂ electrolysis module is approximately 5-7 barg.

“Water electrolysis module,” and “H₂O electrolysis module” refer to a unit capable of splitting H₂O, in the form of steam, into H₂ and O₂ and is defined by the following stoichiometric reaction: 2H₂O+electricity→2H₂+O₂. A water electrolysis module reduces protons to H₂ and oxidizes O²⁻ to O₂. H₂ produced by electrolysis can be blended with a C1-comprising gaseous substrate as a means to supply additional feedstock and to improve substrate composition.

H₂ and CO₂ electrolysis modules have 2 gas outlets. One side of the electrolysis module, the anode, comprises H₂ or CO (and other gases such as unreacted water vapor or unreacted CO₂). The second side, the cathode, comprises O₂ (and potentially other gases). The composition of a feedstock being passed to an electrolysis process may determine the presence of various components in a CO stream. For instance, the presence of inert components, such as CH₄ and/or N₂, in a feedstock may result in one or more of those components being present in the CO-enriched stream. Additionally, in some electrolyzers, O₂ produced at the cathode crosses over to the anode side where CO is generated and/or CO crosses over to the anode side, leading to cross contamination of the desired gas products.

The term “separation module” is used to refer to a technology capable of dividing a substance into two or more components. For example, an “O₂ separation module” may be used to separate an O₂-comprising gaseous substrate into a stream comprising primarily O₂ (also referred to as an “O₂-enriched stream” or “O₂-rich gas”) and a stream that does not primarily comprise O₂, comprises no O₂, or comprises only trace amounts of O₂ (also referred to as an “O₂-lean stream” or “O₂-depleted stream”).

The terms “enriched stream,” “rich gas,” “high purity gas,” and the like refer to a gas stream having a greater proportion of a particular component following passage through a module, such as an rWGS unit, as compared to the proportion of the component in the input stream into the module. For example, a “CO-enriched stream” may be produced upon passage of a CO₂-comprising gaseous substrate through a CO₂ to CO conversion system such as a rWGS unit. An “H₂-enriched stream” may be produced upon passage of a water gaseous substrate through a water electrolysis module. An “O₂-enriched stream” emerges automatically from the anode of a CO₂ or water electrolysis module; an “O₂-enriched stream” may also be produced upon passage of an O₂-comprising gaseous substrate through an O₂ separation module. A “CO₂-enriched stream” may be produced upon passage of a CO₂-comprising gaseous substrate through a CO₂ concentration module.

As used herein, the terms “lean stream,” “depleted gas,” and the like refer to a gas stream having a lesser proportion of a particular component following passage through a module, such as a concentration module or a separation module, as compared to the proportion of the component in the input stream into the module. For example, an O₂-lean stream may be produced upon passage of an O₂-comprising gaseous substrate through an O₂ separation module. The O₂-lean stream may comprise unreacted CO₂ from a CO₂ to CO conversion system. The O₂-lean stream may comprise trace amounts of O₂ or no O₂. A “CO₂-lean stream” may be produced upon passage of a CO₂-comprising gaseous substrate through a CO₂ concentration module. The CO₂-lean stream may comprise CO, H₂, and/or a constituent such as a microbe inhibitor or a catalyst inhibitor. The CO₂-lean stream may comprise trace amounts of CO₂ or no CO₂.

In particular embodiments, the disclosure provides an integrated process wherein the pressure of the gas stream is capable of being increased and/or decreased. The term “pressure module” refers to a technology capable of producing (i.e., increasing) or decreasing the pressure of a gas stream. The pressure of the gas may be increased and/or decreased through any suitable means, for example one or more compressor and/or valve. In certain instances, a gas stream may have a lower than optimum pressure, or the pressure of the gas stream may be higher than optimal, and thus, a valve may be included to reduce the pressure. A pressure module may be located before or after any module described herein. For example, a pressure module may be utilized prior to a removal module, prior to a concentration module, prior to a water electrolysis module, and/or prior to a CO-consuming process.

A “pressurized gas stream” refers to a gaseous substrate that has passed through a pressure module. A “pressurized gas stream” may also be used to refer to a gas stream that meets the operating pressure requirements of a particular module.

The terms “post-CO-consuming process gaseous substrate,” “post-CO-consuming process tail gas,” “tail gas,” and the like may be used interchangeably to refer to a gas that has passed through a CO-consuming process. The post-CO-consuming process gaseous substrate may comprise unreacted CO, unreacted H₂, and/or CO₂ produced (or not taken up in parallel) by the CO-consuming process. The post-CO-consuming process gaseous substrate may further be passed to one or more pressure modules, a removal module, a CO₂ concentration module, and/or a water electrolysis module. In some embodiments, a “post-CO-consuming process gaseous substrate” is a post-fermentation gaseous substrate.

The term “desired composition” is used to refer to the desired level and types of components in a substance, such as, for example, of a gas stream. More particularly, a gas is considered to have a “desired composition” if it contains a particular component (i.e., CO, H₂, and/or CO₂) and/or contains a particular component at a particular proportion and/or does not comprise a particular component (i.e., a contaminant harmful to the microorganisms) and/or does not comprise a particular component at a particular proportion. More than one component may be considered when determining whether a gas stream has a desired composition.

While it is not necessary for the substrate to comprise any H₂, the presence of H₂ should not be detrimental to product formation in accordance with methods of the disclosure. In particular embodiments, the presence of H₂ results in an improved overall efficiency of alcohol production. In one embodiment, the substrate comprises about 30% or less H₂ by volume, 20% or less H₂ by volume, about 15% or less H₂ by volume or about 10% or less H₂ by volume. In other embodiments, the substrate stream comprises low concentrations of H₂, for example, less than 5%, or less than 4%, or less than 3%, or less than 2%, or less than 1%, or is substantially H₂ free.

The substrate may also comprise some CO for example, such as about 1% to about 80% CO by volume, or 1% to about 30% CO by volume. In one embodiment, the substrate comprises less than or equal to about 20% CO by volume. In another embodiment, the substrate comprises less than or equal to about 15% CO by volume, less than or equal to about 10% CO by volume, less than or equal to about 5% CO by volume or substantially no CO.

Substrate composition can be improved to provide a desired or optimum H₂:CO:CO₂ ratio. The desired H₂:CO:CO₂ ratio is dependent on the desired fermentation product of the fermentation process. For ethanol, the optimum H₂:CO:CO₂ ratio would be:

$$( x ) : ( y ) : ( \frac{x - 2 y}{3} ) ,$$

where x>2y, in order to satisfy the stoichiometry for ethanol production:

$$( x ) {H}_{2} + ( y ) CO + ( \frac{x - 2 y}{3} ) {CO}_{2} \to ( \frac{x + y}{6} ) {C}_{2} {H}_{5} OH + ( \frac{x - y}{2} ) {H}_{2} 0 .$$

Operating the fermentation process in the presence of H₂ has the added benefit of reducing the amount of CO₂ produced by the fermentation process. For example, a gaseous substrate comprising minimal H₂ will typically produce ethanol and CO₂ by the following stoichiometry: 6 CO+3H₂O→C₂H₅OH+4 CO₂. As the amount of H₂ utilized by the C1 fixing bacterium increase, the amount of CO₂ produced decreases, i.e., 2 CO+4H₂→C₂H₅OH+H₂O.

When CO is the sole carbon and energy source for ethanol production, a portion of the carbon is lost to CO₂ as follows:

6CO+3H₂O→C₂H₅OH+4CO₂ (ΔG°=−224.90 kJ/mol ethanol)

As the amount of H₂ available in the substrate increases, the amount of CO₂ produced decreases. At a stoichiometric ratio of 1:2 (CO/H₂), CO₂ production is completely avoided.

5CO+1H₂+2H₂O→1C₂H₅OH+3CO₂ (ΔG°=−204.80 kJ/mol ethanol)

4CO+2H₂+1H₂O→1C₂H₅OH+2CO₂ (ΔG°=−184.70 kJ/mol ethanol)

3CO+3H₂→1C₂H₅OH+1CO₂ (ΔG°=−164.60 kJ/mol ethanol)

The composition of the substrate may have a significant impact on the efficiency and/or cost of the reaction. For example, the presence of O₂ may reduce the efficiency of an anaerobic fermentation process. Depending on the composition of the substrate, it may be desirable to treat, scrub, or filter the substrate to remove any undesired impurities, such as toxins, undesired components, or dust particles, and/or increase the concentration of desirable components. Furthermore, carbon capture can be increased by recycling CO₂ produced by a CO-consuming process back to a CO₂ to CO conversion system, thereby improving yield of the CO-consuming process. CO₂ produced by the CO-consuming process may be treated prior to passage through the CO₂ to CO conversion system. In one embodiment the CO₂ to CO conversion system is a rWGS unit, which can be single stage or two or more stages.

In some embodiments, a CO-consuming process is performed in a bioreactor. The term “bioreactor” includes a fermentation device consisting of one or more vessels and/or towers or piping arrangements, which includes the Continuous Stirred Tank Reactor (CSTR), Immobilized Cell Reactor (ICR), Trickle Bed Reactor (TBR), Bubble Column, Gas Lift Fermenter, Static Mixer, a circulated loop reactor, a membrane reactor, such as a Hollow Fiber Membrane Bioreactor (HFM BR) or other vessel or other device suitable for gas-liquid contact. The reactor is preferably adapted to receive a gaseous substrate comprising CO, CO₂, H₂, or mixtures thereof. The reactor may comprise multiple reactors (stages), either in parallel or in series. For example, the reactor may comprise a first growth reactor in which the bacteria are cultured and a second fermentation reactor, to which fermentation broth from the growth reactor may be fed and in which most of the fermentation products may be produced.

Operating a bioreactor at elevated pressures allows for an increased rate of gas mass transfer from the gas phase to the liquid phase. Accordingly, it is generally preferable to perform the culture/fermentation at pressures higher than atmospheric pressure. Also, since a given gas conversion rate is, in part, a function of the substrate retention time and retention time dictates the required volume of a bioreactor, the use of pressurized systems can greatly reduce the volume of the bioreactor required and, consequently, the capital cost of the culture/fermentation equipment. This, in turn, means that the retention time, defined as the liquid volume in the bioreactor divided by the input gas flow rate, can be reduced when bioreactors are maintained at elevated pressure rather than atmospheric pressure. The optimum reaction conditions will depend partly on the particular microorganism used. However, in general, it is preferable to operate the fermentation at a pressure higher than atmospheric pressure. Also, since a given gas conversion rate is in part a function of substrate retention time and achieving a desired retention time in turn dictates the required volume of a bioreactor, the use of pressurized systems can greatly reduce the volume of the bioreactor required, and consequently the capital cost of the fermentation equipment.

Unless the context requires otherwise, the phrases “fermenting,” “fermentation process,” “fermentation reaction” and the like, as used herein, are intended to encompass both the growth phase and product biosynthesis phase of the gaseous substrate. In certain embodiments, the fermentation is performed in the absence of carbohydrate substrates, such as sugar, starch, lignin, cellulose, or hemicellulose.

A culture is generally maintained in an aqueous culture medium that contains nutrients, vitamins, and/or minerals sufficient to permit growth of a microorganism. “Nutrient media,” “nutrient medium,” and “culture medium” are used to describe bacterial growth media. Preferably, the aqueous culture medium is an anaerobic microbial growth medium, such as a minimal anaerobic microbial growth medium. Suitable media are well known in the art. The term “nutrient” includes any substance that may be utilised in a metabolic pathway of a microorganism. Exemplary nutrients include potassium, B vitamins, trace metals, and amino acids.

The terms “fermentation broth” and “broth” are intended to encompass the mixture of components including nutrient media and a culture or one or more microorganisms. It should be noted that the term microorganism and the term bacteria are used interchangeably herein.

A microorganism of the disclosure may be cultured with a gas stream to produce one or more products. For instance, a microorganism of the disclosure may produce or may be engineered to produce ethanol (WO 2007/117157), acetate (WO 2007/117157), butanol (WO 2008/115080 and WO 2012/053905), butyrate (WO 2008/115080), 2,3-butanediol (WO 2009/151342 and WO 2016/094334), lactate (WO 2011/112103), butene (WO 2012/024522), butadiene (WO 2012/024522), methyl ethyl ketone (2-butanone) (WO 2012/024522 and WO 2013/185123), ethylene (WO 2012/026833), acetone (WO 2012/115527), isopropanol (WO 2012/115527), lipids (WO 2013/036147), 3-hydroxypropionate (3-HP) (WO 2013/180581), terpenes, including isoprene (WO 2013/180584), fatty acids (WO 2013/191567), 2-butanol (WO 2013/185123), 1,2-propanediol (WO 2014/036152), 1-propanol (WO 2014/0369152), chorismate-derived products (WO 2016/191625), 3-hydroxybutyrate (WO 2017/066498), 1,3-butanediol (WO 2017/0066498), and 2,3-butanediol (WO2016/094334). In addition to one or more target products, a microorganism of the disclosure may also produce ethanol, acetate, and/or 2,3-butanediol. In certain embodiments, microbial biomass itself may be considered a product. These products may be further converted to produce at least one component of diesel, jet fuel, and/or gasoline. Additionally, the microbial biomass may be further processed to produce a single cell protein (SCP).

A “microorganism” is a microscopic organism, especially a bacterium, archea, virus, or fungus. A microorganism of the disclosure is typically a bacterium. As used herein, recitation of “microorganism” should be taken to encompass “bacterium.”

A “parental microorganism” is a microorganism used to generate a microorganism of the disclosure. The parental microorganism may be a naturally occurring microorganism, known as a wild-type microorganism, or a microorganism that has been previously modified, known as a mutant or recombinant microorganism. A microorganism of the disclosure may be modified to express or overexpress one or more enzymes that were not expressed or overexpressed in the parental microorganism. Similarly, a microorganism of the disclosure may be modified to comprise one or more genes that were not contained by the parental microorganism. A microorganism of the disclosure may also be modified to not express or to express lower amounts of one or more enzymes that were expressed in the parental microorganism. In one embodiment, the parental microorganism is Clostridium autoethanogenum, Clostridium ljungdahlii, or Clostridium ragsdalei. In an embodiment, the parental microorganism is Clostridium autoethanogenum LZ1561, which was deposited on Jun. 7, 2010 with Deutsche Sammlung von Mikroorganismen and Zellkulturen GmbH (DSMZ) located at Inhoffenstraβe 7B, D-38124 Braunschweig, Germany on Jun. 7, 2010 under the terms of the Budapest Treaty and accorded accession number DSM23693. This strain is described in International Patent Application No. PCT/NZ2011/000144, which published as WO 2012/015317.

The term “derived from” indicates that a nucleic acid, protein, or microorganism is modified or adapted from a different, such as a parental or wild-type, nucleic acid, protein, or microorganism, so as to produce a new nucleic acid, protein, or microorganism. Such modifications or adaptations typically include insertion, deletion, mutation, or substitution of nucleic acids or genes. Generally, a microorganism of the disclosure is derived from a parental microorganism. In one embodiment, a microorganism of the disclosure is derived from Clostridium autoethanogenum, Clostridium ljungdahlii, or Clostridium ragsdalei. In an embodiment, a microorganism of the disclosure is derived from Clostridium autoethanogenum LZ1561, which is deposited under DSMZ accession number DSM23693.

A microorganism of the disclosure may be further classified based on functional characteristics. For example, the microorganism of the disclosure may be or may be derived from a C1-fixing microorganism, an anaerobe, an acetogen, an ethanologen, a carboxydotroph, and/or a methanotroph.

“Wood-Ljungdahl” refers to the Wood-Ljungdahl pathway of carbon fixation as described, i.e., by Ragsdale, Biochim Biophys Acta, 1784: 1873-1898, 2008. “Wood-Ljungdahl microorganisms” refers, predictably, to microorganisms comprising the Wood-Ljungdahl pathway. Generally, a microorganism of the disclosure contains a native Wood-Ljungdahl pathway. Herein, a Wood-Ljungdahl pathway may be a native, unmodified Wood-Ljungdahl pathway or it may be a Wood-Ljungdahl pathway with some degree of genetic modification (i.e., overexpression, heterologous expression, knockout, etc.) so long as it still functions to convert CO, CO₂, and/or H₂ to acetyl-CoA.

An “anaerobe” is a microorganism that does not require O₂ for growth. An anaerobe may react negatively or even die if O₂ is present above a certain threshold. However, some anaerobes can tolerate low levels of O₂ (i.e., 0.000001-5% O₂). Typically, a microorganism of the disclosure is an anaerobe.

“Acetogens” are obligately anaerobic bacteria that use the Wood-Ljungdahl pathway as their main mechanism for energy conservation and for synthesis of acetyl-CoA and acetyl-CoA-derived products, such as acetate (Ragsdale, Biochim Biophys Acta, 1784: 1873-1898, 2008). In particular, acetogens use the Wood-Ljungdahl pathway as a (1) mechanism for the reductive synthesis of acetyl-CoA from CO₂, (2) terminal electron-accepting, energy conserving process, (3) mechanism for the fixation (assimilation) of CO₂ in the synthesis of cell carbon (Drake, Acetogenic Prokaryotes, In: The Prokaryotes, 3^{rd} edition, p. 354, New York, N.Y., 2006). All naturally occurring acetogens are C1-fixing, anaerobic, autotrophic, and non-methanotrophic. Typically, a microorganism of the disclosure is an acetogen.

An “ethanologen” is a microorganism that produces or is capable of producing ethanol. Typically, a microorganism of the disclosure is an ethanologen.

An “autotroph” is a microorganism capable of growing in the absence of organic carbon. Instead, autotrophs use inorganic carbon sources, such as CO and/or CO₂. Typically, a microorganism of the disclosure is an autotroph.

A “carboxydotroph” is a microorganism capable of utilizing CO as a sole source of carbon and energy. Typically, a microorganism of the disclosure is a carboxydotroph.

A “methanotroph” is a microorganism capable of utilizing methane as a sole source of carbon and energy. In certain embodiments, a microorganism of the disclosure is a methanotroph or is derived from a methanotroph. In other embodiments, a microorganism of the disclosure is not a methanotroph or is not derived from a methanotroph.

Table 1 provides a representative list of microorganisms and identifies their functional characteristics.

TABLE 1

| Table 1 | Wood-Ljungdahl | C1-fixing | Anaerobe | Acetogen | Ethanologen | Autotroph | Carboxydotroph |
|---|---|---|---|---|---|---|---|
| Acetobacterium woodii | + | + | + | + | +/− ¹ | + | − |
| Alkalibaculum bacchii | + | + | + | + | + | + | + |
| Blautia producta | + | + | + | + | − | + | + |
| Butyribacterium methylotrophicum | + | + | + | + | + | + | + |
| Clostridium aceticum | + | + | + | + | − | + | + |
| Clostridium autoethanogenum | + | + | + | + | + | + | + |
| Clostridium carboxidivorans | + | + | + | + | + | + | + |
| Clostridium coskatii | + | + | + | + | + | + | + |
| Clostridium drakei | + | + | + | + | − | + | + |
| Clostridium formicoaceticum | + | + | + | + | − | + | + |
| Clostridium ljungdahlii | + | + | + | + | + | + | + |
| Clostridium magnum | + | + | + | + | − | + | +/− ² |
| Clostridium ragsdalei | + | + | + | + | + | + | + |
| Clostridium scatologenes | + | + | + | + | − | + | + |
| Eubacterium limosum | + | + | + | + | − | + | + |
| Moorella thermautotrophica | + | + | + | + | + | + | + |
| Moorella thermoacetica (formerly | + | + | + | + | − ³ | + | + |
| Clostridium thermoaceticum) | | | | | | | |
| Oxobacter pfennigii | + | + | + | + | − | + | + |
| Sporomusa ovata | + | + | + | + | − | + | +/− ⁴ |
| Sporomusa silvacetica | + | + | + | + | − | + | +/− ⁵ |
| Sporomusa sphaeroides | + | + | + | + | − | + | +/− ⁶ |
| Thermoanaerobacter kivui | + | + | + | + | − | + | − |

¹ Acetobacterium woodii can produce ethanol from fructose, but not from gas.
² It has not been investigated whether Clostridium magnum can grow on CO.
³ One strain of Moorella thermoacetica, Moorella sp. HUC22-1, has been reported to produce ethanol from gas.
⁴ It has not been investigated whether Sporomusa ovata can grow on CO.
⁵ It has not been investigated whether Sporomusa silvacetica can grow on CO.
⁶ It has not been investigated whether Sporomusa sphaeroides can grow on CO.

A “native product” is a product produced by a genetically unmodified microorganism. For example, ethanol, acetate, and 2,3-butanediol are native products of Clostridium autoethanogenum, Clostridium ljungdahlii, and Clostridium ragsdalei. A “non-native product” is a product that is produced by a genetically modified microorganism but is not produced by a genetically unmodified microorganism from which the genetically modified microorganism is derived.

“Selectivity” refers to the ratio of the production of a target product to the production of all fermentation products produced by a microorganism. A microorganism of the disclosure may be engineered to produce products at a certain selectivity or at a minimum selectivity. In one embodiment, a target product account for at least about 5%, 10%, 15%, 20%, 30%, 50%, or 75% of all fermentation products produced by a microorganism of the disclosure. In one embodiment, the target product accounts for at least 10% of all fermentation products produced by a microorganism of the disclosure, such that a microorganism of the disclosure has a selectivity for the target product of at least 10%. In another embodiment, the target product accounts for at least 30% of all fermentation products produced by a microorganism of the disclosure, such that a microorganism of the disclosure has a selectivity for the target product of at least 30%.

A culture/fermentation should desirably be carried out under appropriate conditions for production of the target product. Typically, the culture/fermentation is performed under anaerobic conditions. Reaction conditions to consider include pressure (or partial pressure), temperature, gas flow rate, liquid flow rate, media pH, media redox potential, agitation rate (if using a continuous stirred tank reactor), inoculum level, maximum gas substrate concentrations to ensure that gas in the liquid phase does not become limiting, and maximum product concentrations to avoid product inhibition. In particular, the rate of introduction of the substrate may be controlled to ensure that the concentration of gas in the liquid phase does not become limiting, since products may be consumed by the culture under gas-limited conditions.

Target products may be separated or purified from a fermentation broth using any method or combination of methods known in the art, including, for example, fractional distillation, evaporation, pervaporation, gas stripping, phase separation, and extractive fermentation, including for example, liquid-liquid extraction. In certain embodiments, target products are recovered from the fermentation broth by continuously removing a portion of the broth from the bioreactor, separating microbial cells from the broth (conveniently by filtration), and recovering one or more target products from the broth. Alcohols and/or acetone may be recovered, for example, by distillation. Acids may be recovered, for example, by adsorption on activated charcoal. Separated microbial cells are preferably returned to the bioreactor. The cell-free permeate remaining after target products have been removed is also preferably returned to the bioreactor. Additional nutrients (such as B vitamins) may be added to the cell-free permeate to replenish the medium before it is returned to the bioreactor.

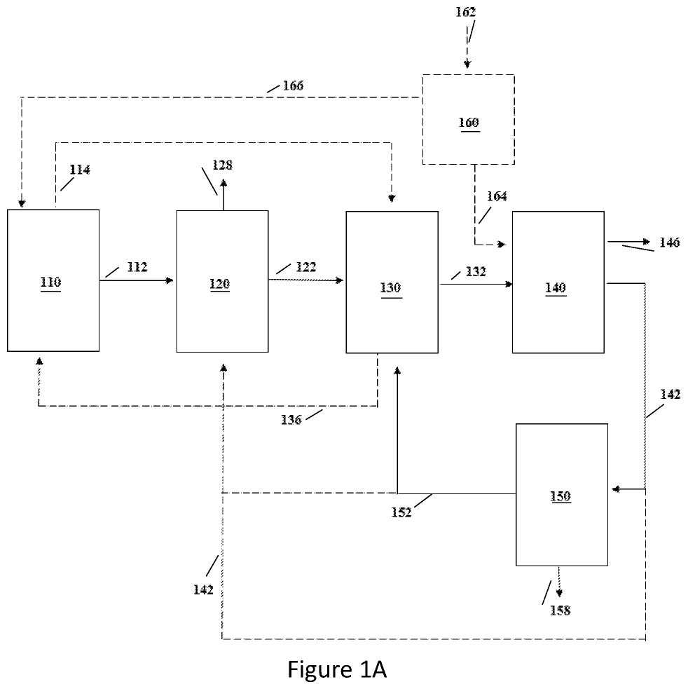

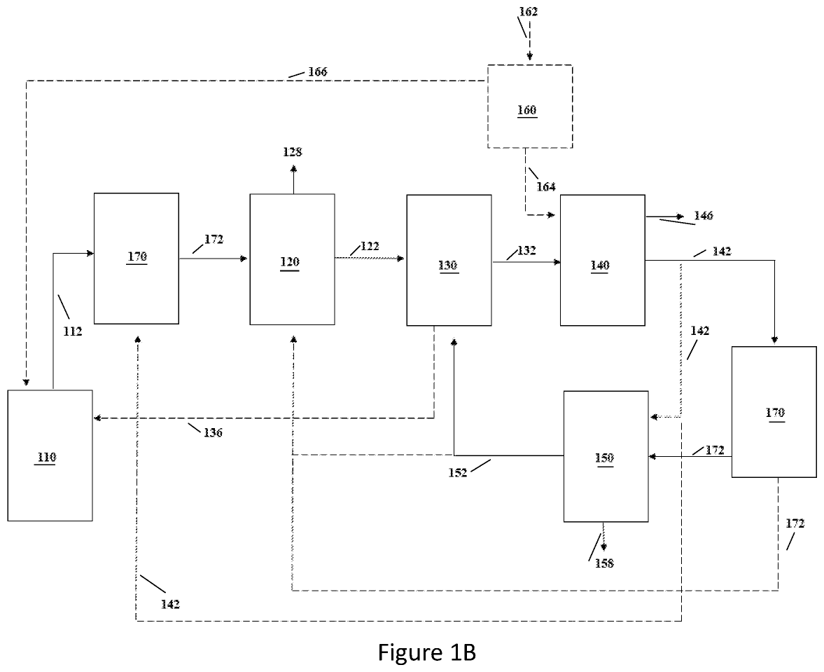

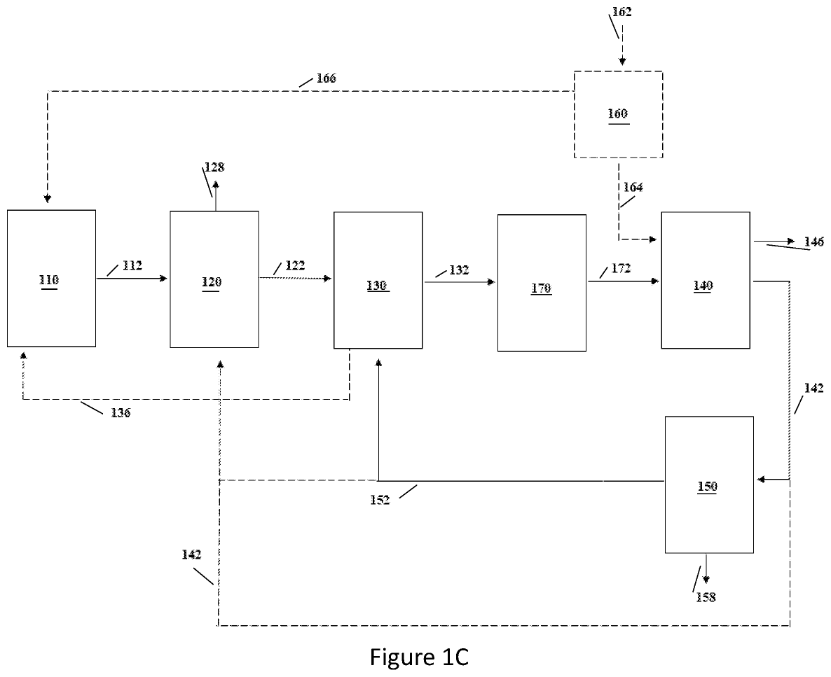

FIG. 1A shows a process for integration of an industrial process 110, one or more removal module 120, a CO₂ to CO conversion system 130, an optional water electrolysis process 160, and a CO-consuming process 140. CO₂-comprising gas from an industrial process 110 is fed via a conduit 112 to one or more removal module 120 to remove and/or convert one or more constituent 128. In one embodiment CO₂ to CO conversion system 130 is a rWGS unit. In one embodiment the rWGS unit has a single stage. In one embodiment the rWGS unit has at least two stages. The treated gas from the one or more removal modules 120 is then fed via a conduit 122 to CO₂ to CO conversion system 130 for conversion of at least a portion of the gas stream. In some embodiments, CO₂-comprising gas from the industrial process 110 is directly fed via a conduit 114 to CO₂ to CO conversion system 130 for conversion of at least a portion of the gas stream; in this embodiment, a constituent such as sulfur-comprising compound may be removed prior to passage through an industrial process. Optionally, at least a portion of the H₂O perhaps in the form of vapor or steam that is generated as a product of the reverse water gas shift reaction may be recycled from the CO₂ to CO conversion system 130 to the industrial process 110 via a conduit 136. At least a portion of the converted gas stream is passed, via a conduit 132, from the CO₂ to CO conversion system 130, which in this example is a rWGS unit, to a CO-consuming process 140. In some embodiments, a water substrate is fed via a conduit 162 to a water electrolysis module 160 for conversion of at least a portion of the water substrate, and an H₂-enriched stream is passed via a conduit 164 to the CO-consuming process 140. Depending upon the selected CO₂ to CO conversion system 130, second H₂-enriched stream 163 from water electrolysis module 160 may be passed to CO₂ to CO conversion system 130. For example, if CO₂ to CO conversion system is a rWGS unit, second H₂-enriched stream 163 from water electrolysis module 160 is passed to CO₂ to CO conversion system 130. FIG. 1A shows second H₂-enriched stream 163 as branching from H₂-enriched stream 164, however in other embodiments second H₂-enriched stream 163 may be independent from H₂-enriched stream 164. Optionally, at least a portion of O₂ generated by the water electrolysis module 160 may be passed to the industrial process 110 via a conduit 166. The CO-consuming process 140 produces at least one product 146 and a post-CO-consuming process gaseous substrate 142.

The CO-consuming process 140 of FIG. 1A may be a gas fermentation process and may occur in an inoculator and/or one or more bioreactors. For example, the CO-consuming process 140 may be a gas fermentation process in a bioreactor comprising a culture of at least one C1-fixing microorganism. In embodiments wherein the CO-consuming process 140 is a gas fermentation process, a culture may be fermented to produce one or more fermentation products 146 and a post-fermentation gaseous substrate, such as CO-consuming process gaseous substrate 142.

In some embodiments, the CO-consuming process 140 of FIG. 1A comprises a CO₂-producing reaction step. In embodiments wherein a post-CO-consuming process gaseous substrate 142 comprises CO₂, at least a portion of the post-CO-consuming process gaseous substrate 142 is passed to one or more removal modules 150 to remove and/or convert one or more constituent 158. A treated gas stream comprising CO₂ 152 is then passed to CO₂ to CO conversion system 130 for conversion of at least a portion of treated gas stream comprising CO₂ 152 or treated gas stream comprising CO₂ 152 may be passed to the one or more removal modules 120 that receives the CO₂-comprising gas 112 from the industrial process 110. In some embodiments, the post-CO-consuming process gaseous substrate 142 is passed to the same one or more removal modules 120 that receives CO₂-comprising gas 112 from the industrial process 110. In various embodiments, the post-CO-consuming process gaseous substrate 142 may be passed to the one or more removal modules 120 that receives the CO₂-comprising gas 112 from the industrial process 110 This process of treating and converting CO₂ to CO of the post-CO-consuming process gaseous substrate has been found to increase carbon capture efficiency.

In particular embodiments, at least one constituent removed by the removal module 150 of FIG. 1A is produced, introduced, and/or concentrated by the CO-consuming process 140, such as a gas fermentation process. In various embodiments, the one or more constituent produced, introduced, and/or concentrated by the fermentation step comprises sulfur-comprising compounds. In certain instances, sulfur-comprising compounds, such as hydrogen sulfide, is introduced to the CO-consuming process 140. This sulfur (present as sulfur-comprising compounds) was found to reduce the efficiency of the CO₂ to CO conversion system 130. For example, sulfur-comprising compounds may harm one or more catalysts used in different rWGS processes employed in specific embodiments as the CO₂ to CO conversion system The one or more removal modules 150 was found to be successful at reducing the amount of sulfur-comprising compounds in the post-CO-consuming process gaseous substrate prior to the post-CO-consuming process gaseous substrate being passed to the CO₂ to CO conversion system 130. The use of the removal module 150 prior to the CO₂ to CO conversion system 130 was found to increase the efficiency of the CO₂ to CO conversion system 130.

The O₂ by-product of water electrolysis processes employed, for example when the CO₂ to CO conversion process is a rWGS unit, can provide additional benefits for the C1-generating industrial process, discussed above. Specific embodiments of the fermentation processes of the current disclosure are anaerobic processes, and depending upon the technology selected for the CO₂ to CO conversion system, O₂ could be generated as a by-product and may be separated and passed through optional conduit 136 in of FIG. 1A, to be used in the industrial process 110. The optional O₂ by-product 136 of the CO₂ to CO conversion process 130 can be integrated with the industrial process 110 and beneficially offset costs, and in some cases, have synergy that further reduces costs for both the industrial process 110 as well as the subsequent gas fermentation. In some embodiments, the CO₂ to CO conversion system will not generate O₂ as a by-product.

Typically, the industrial processes described herein derive the required O₂ by air separation. Production of O₂ by air separation is an energy intensive process which involves cryogenically separating O₂ from N₂ to achieve the highest purity. Production of O₂ by CO₂ conversion to CO as in line 136, depending upon the CO₂ to CO conversion system selected, and/or water electrolysis as in line 166, and displacing O₂ produced by air separation, could offset up to 5% of the electricity costs in an industrial process.

Several C1-generating industrial processes involving partial oxidation reactions require an O₂ input. Exemplary industrial processes include Basic Oxygen Furnace (BOF) reactions, COREX or FINEX steel making processes, Blast Furnace (BF) processes, ferroalloy production processes, non-ferrous products manufacturing, petroleum refining, petrochemical production, carbohydrate fermentation, cement making, titanium dioxide production processes, gasification processes and any combinations thereof. Gasification processes include, but are not limited to, gasification of coal, gasification of refinery residues, gasification of biomass, gasification of lignocellulosic material, black liquor gasification, gasification of municipal solid waste, gasification of industrial solid waste, gasification of sewerage, gasification of sludge from wastewater treatment, gasification of pet coke, reforming of natural gas, reforming of biogas, reforming of landfill gas or any combination thereof. In one or more of these industrial processes, O₂ from the CO₂ to CO conversion system and/or O₂ from water electrolysis may be used to off-set or completely replace the O₂ typically supplied through air separation.

As shown in FIGS. 1B and 1C, a process for integration of an industrial process, one or more removal module, a CO₂ to CO conversion system, an optional water electrolysis process, and a CO-consuming process may further comprise integration of one or more pressure modules 170. For example, as shown in FIG. 1B, at least a portion of CO₂-comprising gas 112 from an industrial process 110 is passed to pressure module 170 to produce a pressurized CO₂-comprising gas stream 172. At least a portion of the pressurized CO₂-comprising gas stream 172 is then passed to a removal module 120. At least a portion of post-CO-consuming process gaseous substrate 142 may also be passed pressure module 170 to produce a pressurized tail gas which is part of pressurized CO₂-comprising gas stream 172. As shown in FIG. 1C, at least a portion of a converted gas stream 132 is passed from CO₂ to CO conversion system 130 to pressure modules 170 to produce pressurized CO-comprising gas stream 172, which is passed CO-consuming process 140.

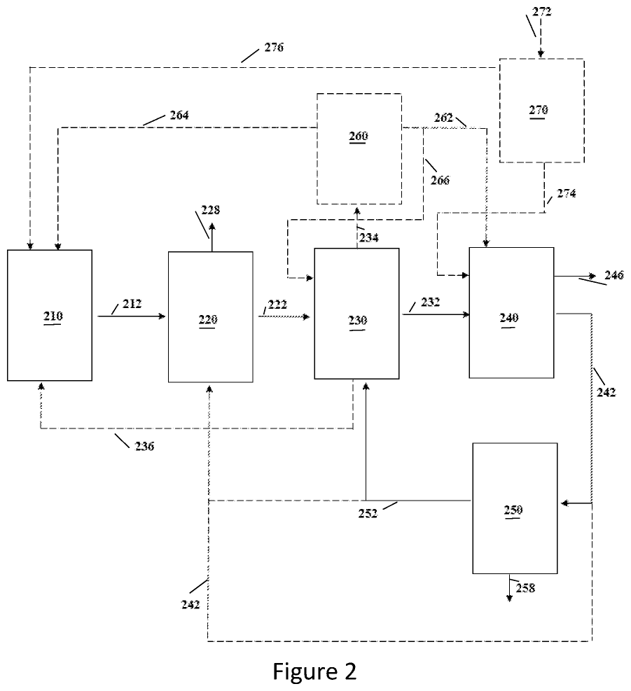

FIG. 2 shows a process for integration of an industrial process 210, a removal module 220, a CO₂ to CO conversion system 230, an optional water electrolysis process 270, a CO-consuming process 240, and an optional O₂ separation module 260. In FIG. 2, the CO₂ to CO conversion system 230 is selected to be a rWGS unit. CO₂-comprising gas 212 from an industrial process 210 is passed to one or more removal modules 220 to remove and/or convert one or more constituent 228. The treated gas 222 from the one or more removal module 220 is then passed CO₂ to CO conversion system 230 for conversion of at least a portion of the CO₂ in treated gas stream 222. If the selected CO₂ to CO conversion system generates O₂, optionally, at least a portion of O₂ may be fed from the CO₂ to CO conversion system 230 to the industrial process 210 via a conduit 236. At least a portion of the converted gas stream 232 is passed from the CO₂ to CO conversion system 230 to the CO-consuming process 240 to produce a product 246 and a post-CO-consuming process gaseous substrate 242. In some embodiments, a water substrate 272 is introduced to water electrolysis module 270 for conversion of at least a portion of the water substrate to generate an H₂-enriched stream 274 which is passed to the CO-consuming process 240. If necessary, a portion of H₂-enriched stream 274 may be passed in stream 273 to CO₂ to CO conversion system 230. Optionally, at least a portion of O₂ generated by water electrolysis module 270 may be passed in O₂ stream 276 to the industrial process 210.

In particular embodiments where the CO₂ to CO conversion system generates O₂ by-product, the process includes an O₂ separation module 260 following the CO₂ to CO conversion system 230 to separate at least a portion of O₂ from the gas generated in CO₂ to CO conversion system 230. In embodiments utilizing an O₂ separation module 260 downstream of CO₂ to CO conversion system 230, at least a portion of gas stream 234 is fed from the CO₂ to CO conversion system 230 to O₂ separation module 260. In embodiments incorporating O₂ separation module 260, an O₂-enriched stream 264 may be passed industrial process 210 thereby displacing the need for other sources of O₂ in industrial process 210. In embodiments utilizing O₂ separation module 260 downstream of CO₂ to CO conversion system 230, at least a portion of the O₂-lean stream 262 is passed from O₂ separation module 260 to the CO-consuming process 240. In some embodiments utilizing an O₂ separation module 260 downstream of CO₂ to CO conversion system 230, at least a portion of the O₂-lean stream 262 is passed from O₂ separation module 260 back to the CO₂ to CO conversion system 230 in line 266. In embodiments not utilizing an O₂ separation module 260, a portion of the gas stream 236 may be passed from the CO₂ to CO conversion system 230 to the industrial process 210.

In some embodiments, the CO-consuming process 240 of FIG. 2 comprises a CO₂-producing reaction step. In embodiments wherein the post-CO-consuming process gaseous substrate comprises CO₂, at least a portion of the post-CO-consuming process gaseous substrate is passed via a conduit 242 to one or more removal module 250 to remove and/or convert one or more constituent 258. A treated gas stream 252 is then passed CO₂ to CO conversion system 230 for conversion of at least a portion of the treated gas stream 252. In particular embodiments, the post-CO-consuming process gaseous substrate 242 is passed to the same one or more removal module 220 that receives the CO₂-comprising gas 212 from the industrial process 210. In various embodiments, the post-CO-consuming process gaseous substrate 242 and 252 may be passed to the one or more removal modules 220 that receives the CO₂-comprising gas 212 from the industrial process 210 and the one or more removal modules 250.

The CO-consuming process 240 of FIG. 2 may be a gas fermentation process and may occur in an inoculator and/or one or more bioreactors. For example, the CO-consuming process 240 may be a gas fermentation process in a bioreactor comprising a culture of at least one C1-fixing microorganism. In embodiments wherein the CO-consuming process 240 is a gas fermentation process, a culture may be fermented to produce one or more fermentation products such as post CO-consuming process product 246 and a post-fermentation gaseous substrate such as the post-CO-consuming process gaseous substrate 242.

Providing a high purity CO₂ stream, a CO₂-rich stream, to a CO₂ to CO conversion system, such as a rWGS unit, increases the carbon capture efficiency of a CO-consuming process. To increase the concentration of CO₂ in a stream, one or more CO₂ concentration module may be incorporated in the process. The CO-enriched stream generated by the CO₂ to CO conversion system, such as a rWGS unit, stream may have a concentration of CO between 20-90%.

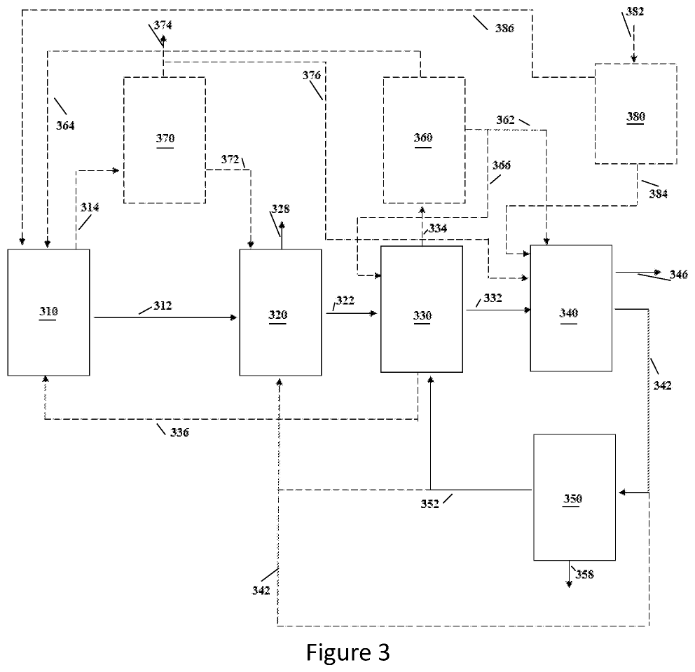

FIG. 3 shows a process for integration of an industrial process 310 with an optional CO₂ concentration module 370, a removal module 320, a CO₂ to CO conversion system 330, an optional water electrolysis module 380, a CO-consuming process 340, and an optional O₂ separation module 360, in accordance with one aspect of the disclosure. In embodiments not including the CO₂ concentration module 370, CO₂-comprising gas 312 from the industrial process 310 is passed to a removal module 320. In embodiments including CO₂ concentration module 370, CO₂-comprising gas 314 from the industrial process 310 is passed to CO₂ concentration module 370 in order to increase the concentration of CO₂ in the gas stream and to remove one or more constituent 374. The CO₂-concentrated gas stream 372 is passed to one or more removal modules 320 to remove and/or convert one or more constituent 328. The treated gas 322 from the one or more removal module 320 is then passed to CO₂ to CO conversion system 330 for conversion of at least a portion of treated gas stream 322. CO₂ to CO conversion system 330 may be a rWGS unit. At least a portion of converted gas stream 332 is passed from the CO₂ to CO conversion system 330 to CO-consuming process 340. In some embodiments, the constituent 374 is CO and/or H₂, which is passed via conduit 376 to CO-consuming process 340. In some embodiments, a water substrate 382 is fed to water electrolysis module 380 for conversion of at least a portion of water substrate 382, to generate H₂-enriched stream 384 which is passed to CO-consuming process 340. Depending upon the CO₂ to CO conversion system selected, such as a rWGS unit which uses H₂ as a reactant, a portion of H₂-enriched stream 384 may be passed to CO₂ to CO conversion system 330 in stream 383. Of course, an independent H₂-enriched stream may be passed from water electrolysis module 380 to CO₂ to CO conversion system in lieu of or in addition to stream 383 (not shown). Optionally, at least a portion of O₂-enriched stream 386 generated by water electrolysis module 380 may be passed to industrial process 310.

At least a portion of the gas stream 336 from the CO₂ to CO conversion system 330 may be passed to the industrial process 310. In particular embodiments, the process includes an O₂ separation module 360 following the CO₂ to CO conversion system 330, where the gas stream 334 is passed from the CO₂ to CO conversion system 330 to the O₂ separation module 360 to separate at least a portion of O₂ from the gas stream 334. In embodiments utilizing O₂ separation module 360 after the CO₂ to CO conversion system 330, at least a portion of the O₂-enriched stream 364 is passed from O₂ separation module 360 to industrial process 310. In embodiments utilizing an O₂ separation module 360 after the CO₂ to CO conversion system 330, at least a portion of the O₂-lean stream 362 is passed from O₂ separation module 360 to CO-consuming process 340. In some embodiments utilizing an O₂ separation module 360 after the CO₂ to CO conversion system 330, at least a portion of the O₂-lean stream 366 is passed from the O₂ separation module 260 back to CO₂ to CO conversion system 330. In embodiments not utilizing an O₂ separation module 360, a portion of the gas stream 336 may be passed from the CO₂ to CO conversion system 330 to industrial process 310.

Concentrating the CO₂ in the gas stream 314 prior to the one or more removal modules 320 decreases undesired gases and thereby increases the efficiency of the CO-consuming process 340, which may be a gas fermentation process.

In some embodiments, the CO-consuming process 340 of FIG. 3 comprises a CO₂-producing reaction step. In embodiments wherein the post-CO-consuming process gaseous substrate comprises CO₂, the post-CO-consuming process gaseous substrate 342 is passed to one or more removal modules 350 to remove and/or convert one or more constituent 358. The treated gas stream 352 is then passed to CO₂ to CO conversion system 330 for conversion of at least a portion of treated gas stream 352. In particular embodiments, the post-CO-consuming process gaseous substrate 342 is passed to the one or more removal modules 320 that receives the CO₂-comprising gas 312 and or 372 from industrial process 310. In various embodiments, the post-CO-consuming process gaseous substrate 342 and 352 may be passed to the one or more removal modules 320 that receives the CO₂-comprising gas 312 and or 372 from industrial process 310 and one or more removal modules 350.

The CO-consuming process 340 of FIG. 3 may be a gas fermentation process and may occur in an inoculator and/or one or more bioreactors. For example, the CO-consuming process may be a gas fermentation process in a bioreactor comprising a culture of at least one C1-fixing microorganism. In embodiments wherein the CO-consuming process 340 is a gas fermentation process, a culture may be fermented to produce one or more fermentation products such as post CO-consuming process product 346 and a post-fermentation gaseous substrate, such as the post-CO-consuming process gaseous substrate 342.

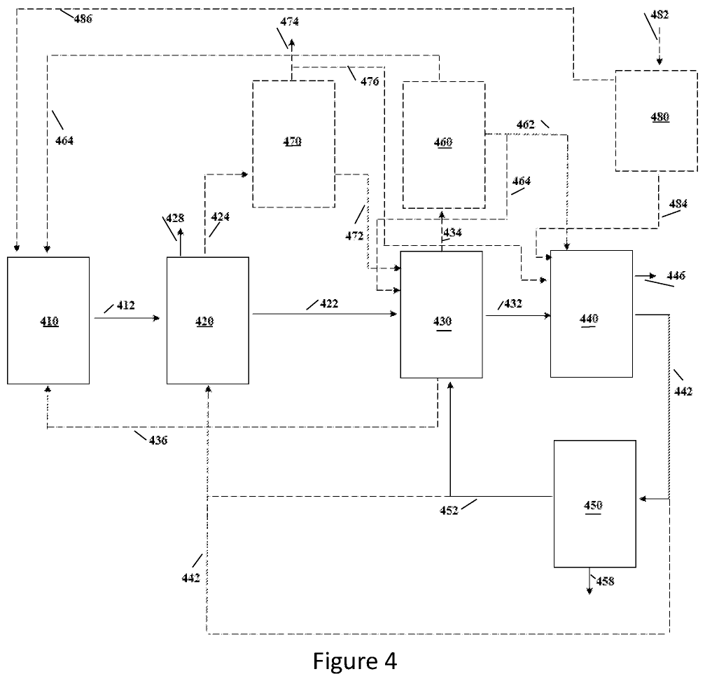

In particular embodiments, a CO₂ concentration module may be placed after a removal module. FIG. 4 shows a process for integration of an industrial process 410 with a removal module 420, an optional CO₂ concentration module 470, a CO₂ to CO conversion system 430, an optional water electrolysis module 480, a CO-consuming process 440, and an optional O₂ separation module 460, in accordance with one aspect of the disclosure. In embodiments not including an optional CO₂ concentration module 470, CO₂-comprising gas 422 from the industrial process 410 is passed from removal module 420 to the CO₂ to CO conversion system 430. In embodiments including optional CO₂ concentration module 470, CO₂-comprising gas 412 from the industrial process 410 is passed to one or more removal modules 420 to remove and/or convert one or more constituent 428. Resulting treated stream 424 is then passed to optional CO₂ concentration module 470 in order to increase the concentration of the CO₂ in CO₂-concentrated gas stream 472 and remove one or more constituent 474. CO₂-concentrated gas stream 472 is then passed CO₂ to CO conversion system 430 for conversion of at least a portion of the gas stream. At least a portion of the converted gas stream 432 may be passed from the CO₂ to CO conversion system 430 to the CO-consuming process 440. In some embodiments, the constituent 474 is CO and/or H₂, which is passed via conduit 476 to CO-consuming process 440. In some embodiments, water substrate 482 is fed to water electrolysis module 480 for conversion of at least a portion of water substrate 482, to generate H₂-enriched stream 484 is passed to CO-consuming process 440. Depending upon the CO₂ to CO conversion system selected, such as a rWGS unit which uses H₂ as a reactant, a portion of H₂-enriched stream 484 may be passed to CO₂ to CO conversion system 430 in stream 483. Of course, an independent H₂-enriched stream may be passed from water electrolysis module 480 to CO₂ to CO conversion system in lieu of or in addition to stream 483 (not shown). Optionally, at least a portion of O₂-enriched stream 486 generated by water electrolysis module 480 may be passed to industrial process 410.

At least a portion of the gas stream 436 from the CO₂ to CO conversion system 430 may be passed to the industrial process 410. In particular embodiments, the process includes O₂ separation module 460 following the CO₂ to CO conversion system 430 to separate at least a portion of O₂ from the gas stream 434. In embodiments utilizing an O₂ separation module 460 after the CO₂ to CO conversion system 430, at least a portion of the gas stream 464 is fed from the O₂ separation module 460 to the industrial process 410. In embodiments utilizing O₂ separation module 460 after the CO₂ to CO conversion system 430, at least a portion of the O₂-lean stream 462 is passed from O₂ separation module 460 to CO-consuming process 440. In some embodiments utilizing O₂ separation module 460 after CO₂ to CO conversion system 430, at least a portion of the O₂-lean stream 466 is passed from the O₂ separation module 460 back to the CO₂ to CO conversion system 430. In embodiments not utilizing O₂ separation module 460, a portion of the gas stream 436 may be passed from the CO₂ to CO conversion system 430 to the industrial process 410, particularly if the selected CO₂ to CO conversion system 430 generates O₂.

In some embodiments, the CO-consuming process 440 of FIG. 4 comprises a CO₂-producing reaction step. In embodiments wherein a post-CO-consuming process gaseous substrate comprises CO₂, at least a portion of the post-CO-consuming process gaseous substrate 442 is passed to one or more removal modules 450 to remove and/or convert one or more constituents 458. The treated gas stream 452 is then passed to CO₂ to CO conversion system 430 for conversion of at least a portion of the treated gas stream 452. In particular embodiments, the post-CO-consuming process gaseous substrate 442 is passed to the same one or more removal modules 420 that receives the CO₂-comprising gas 412 from the industrial process 410. In various embodiments, the post-CO-consuming process gaseous substrate 442 and 452 may be passed to the one or more removal modules 420 that receives the CO₂-comprising gas from the industrial process 410 and one or more removal modules 450.

The CO-consuming process 440 of FIG. 4 may be a gas fermentation process and may occur in an inoculator and/or one or more bioreactors. For example, the CO-consuming process 440 may be a gas fermentation process in a bioreactor comprising a culture of at least one C1-fixing microorganism. In embodiments wherein the CO-consuming process 440 is a gas fermentation process, a culture may be fermented to produce one or more fermentation products such as post CO-consuming process product 446 and a post-fermentation gaseous substrate, such as the post-CO-consuming process gaseous substrate 442.

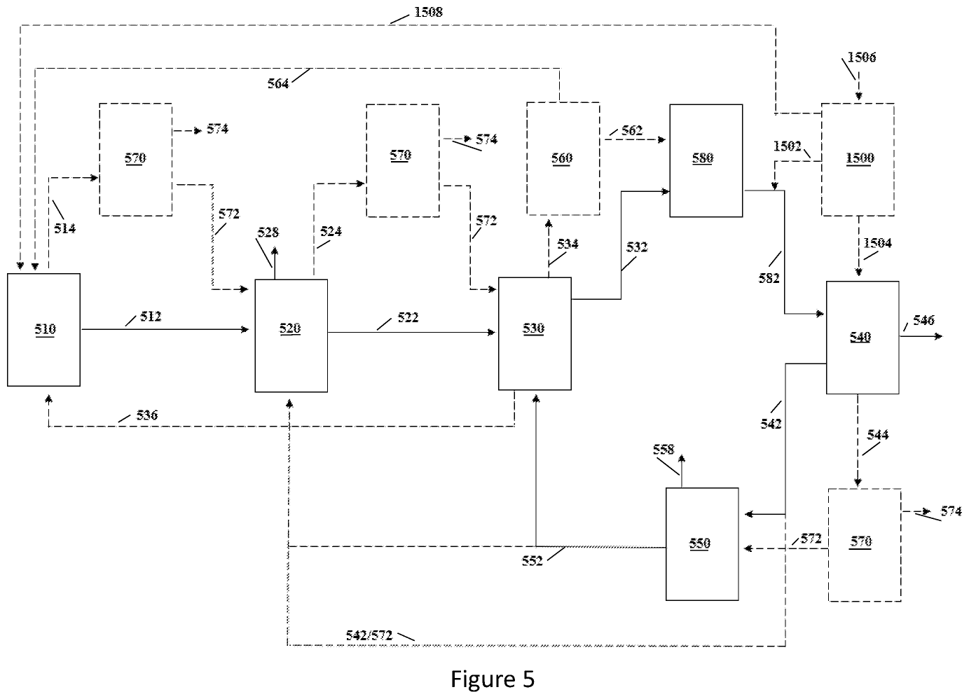

FIG. 5 shows a process for integration of an industrial process 510 with a removal module 520, optional CO₂ concentration modules 570, a CO₂ to CO conversion system 530, a CO-consuming process 540, an optional O₂ separation module 560, an optional pressure module 580, and an optional water electrolysis module 1500, in accordance with one aspect of the disclosure. CO₂-comprising gas 512 from the industrial process 510 is passed to one or more removal modules 520 to remove and/or convert one or more constituent 528. The treated gas 522 from the one or more removal module 520 is then passed to CO₂ to CO conversion system 530 for conversion of at least a portion of the gas stream 522. In embodiments that blend H₂, a water electrolysis module 1500 may generate and pass a H₂-rich gas stream 1502 to be blended with the optionally pressurized converted gas stream 582 prior to being introduced to the CO-consuming process 540.

In particular embodiments, the disclosure provides one or more pressure modules 580 to increase the pressure of the converted gas 532 from the CO₂ to CO conversion system 530. In embodiments utilizing a pressure module 580 after the CO₂ to CO conversion system 530, at least a portion of the gas stream 532 is passed from CO₂ to CO conversion system 530 to pressure module 580 which increases the pressure of gas stream 532 and generates increased pressure stream 582 which is passed to CO-consuming process 540.

In various embodiments, water electrolysis module 1500 is incorporated along with the O₂ separation module 560 and/or the pressure module 580. In various embodiments, a water substrate 1506 is introduced to water electrolysis module 1500, and H₂-rich gas stream 1502 is blended with the converted gas stream 582 prior to converted gas stream 582 being introduced to CO-consuming process 540. In various embodiments, H₂-rich gas stream 1504 is passed directly from water electrolysis module 1500 to CO-consuming process 540. Depending upon the CO₂ to CO conversion system selected, such as a rWGS unit which uses H₂ as a reactant, an H₂-enriched stream 1510 may be passed from water electrolysis module 1500 to CO₂ to CO conversion system 530. Optionally, at least a portion of O₂-enriched stream 1508 generated by water electrolysis module 1500 may be passed to industrial process 510.

In certain embodiments, the disclosure integrates an industrial process 510, an optional CO₂ concentration module 570, a removal module 520, a CO₂ to CO conversion system 530, an optional O₂ separation module 560, an optional pressure module 580, an water electrolysis module 1500, and a CO-consuming process 540, in accordance with one aspect of the disclosure. CO₂-comprising gas 514 from the industrial process 510 is passed to an optional CO₂ concentration module 570 to increase the concentration of the CO₂ in the gas stream 514 and remove one or more constituent 574. A first CO₂ concentrated stream 572 from first CO₂ concentration module 570 is passed to removal module 520 to remove and/or convert one or more constituent 528. The treated stream 524 is then passed to a second optional CO₂ concentration module 570 to increase the concentration of the CO₂ in the gas stream 524 and remove one or more constituent 574. A second CO₂ concentrated stream 572 is passed to a CO₂ to CO conversion system 530 for conversion of at least a portion of the second CO₂ concentrated stream 572. At least a portion of the converted gas stream 534 may be passed to an optional O₂ separation module 560 to separate at least a portion of O₂ from the converted gas stream 534. At least a portion of the O₂-rich gas stream 564 may be passed from the optional O₂ separation module 560 to the industrial process 510. At least a portion of the O₂-rich gas stream may be fed from the CO₂ to CO conversion system 530 to the industrial process 510 via a conduit 536, if the selected CO₂ to CO conversion system 530 generates O₂. At least a portion of the O₂-depleted gas stream 562 may be passed from the optional O₂ separation module 560 to an optional pressure module 580. The pressurized gas stream 582 from the optional pressure module 580 is passed to the CO-consuming process 540. The pressurized gas stream 582 may be blended with an H₂-rich gas stream 1502 prior to being introduced to the CO-consuming process 540.

The CO-consuming process 540 of FIG. 5 produces product 546 and post-CO-consuming process gaseous substrate 542. The CO-consuming process may be a gas fermentation process and may occur in an inoculator and/or one or more bioreactors. In embodiments wherein the CO-consuming process 540 is a gas fermentation process, a culture may be fermented to produce one or more fermentation products such as post CO-consuming process product 546 and a post-fermentation gaseous substrate, such as the post-CO-consuming process gaseous substrate 542 and or 544. The post-CO-consuming process gaseous substrate 542 may be passed removal module 550 to remove and/or convert one or more constituent 558. In embodiments including a CO₂ concentration module 570 after the CO-consuming process, the post-CO-consuming process gaseous substrate 544 may be passed to an optional CO₂ concentration module 570 to increase the concentration of the CO₂ in stream 544 and remove one or more constituent 574. Resulting CO₂-enriched stream 572 is passed to removal module 550 to remove and/or convert one or more constituent 558. The treated gas stream 552 may then be passed to CO₂ to CO conversion system 530 for conversion of at least a portion of the gas stream 552. In particular embodiments, post-CO-consuming process gaseous substrate 542 is passed, to the same one or more removal modules 520 that receives the CO₂-comprising gas 512 from the industrial process 510. In various embodiments, the post-CO-consuming process gaseous substrate 542 may be passed to both the one or more removal modules 520 that receives the CO₂-comprising gas 512 or 572 from the industrial process 510 and the one or more removal module 550.

The disclosure provides generally for the removal of constituents from the gas stream that may have adverse effects on downstream processes, for instance, the downstream fermentation process and/or downstream modules. In particular embodiments, the disclosure provides for one or more further removal module between the various modules in order to prevent the occurrence of such adverse effects.

In various instances, the conversion of a CO₂-comprising gaseous substrate by an CO₂ to CO conversion system results in one or more constituent passing through the CO₂ to CO conversion system 630. In various embodiments, this results in one or more constituent in the CO-enriched stream. In certain instances, the constituent includes portions of converted O₂. In various embodiments, the further removal module is a deoxygenation module for removing O₂ from the CO-enriched stream.

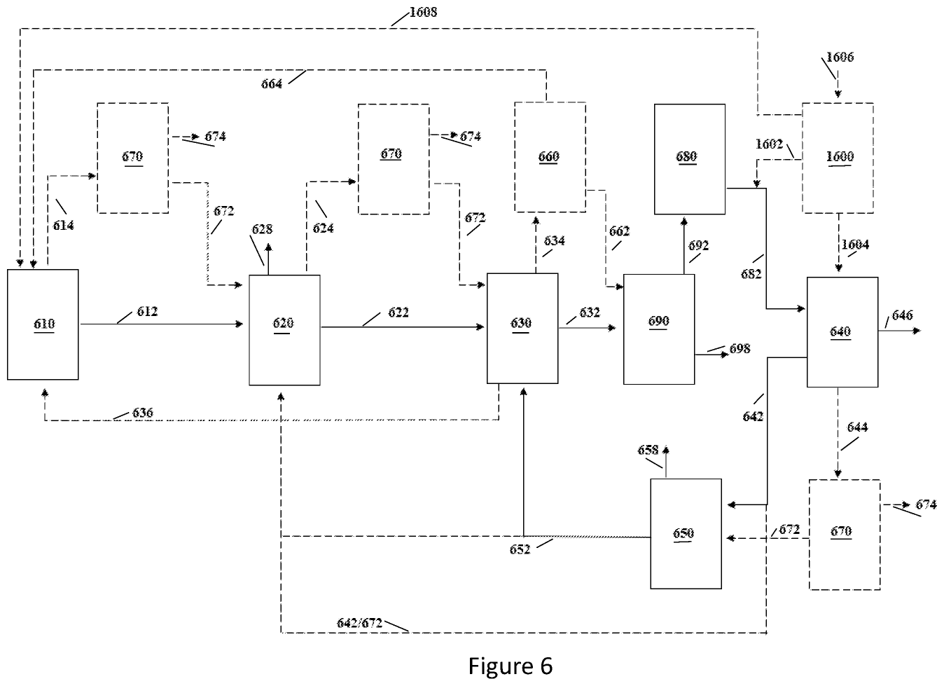

FIG. 6 shows the integration of a CO₂ to CO conversion system 630, an optional O₂ separation module 660, an optional pressure module 680, with a further removal module 690. In certain instances, the further removal module 690 is downstream of the CO₂ to CO conversion system 630. In embodiments where the further removal module 690 is downstream of the CO₂ to CO conversion system 630, at least a portion of the gas stream 632 from the CO₂ to CO conversion system 630 is passed to the further removal module 690. The further removal module 690 removes and/or converts one or more constituents 698 in gas stream 632. Additionally, I some embodiments, when utilizing an optional O₂ separation module 660, stream 662 from optional O2 separation module 660 is passed to further removal module 690 to remove and/or convert one or more constituents 698. The treated stream 692 is then passed to an optional pressure module 680.

In certain embodiments, the disclosure integrates an industrial process 610, an optional CO₂ concentration module 670, a removal module 620, a CO₂ to CO conversion system 630, a further removal module 690, an optional O₂ separation module 660, an optional pressure module 680, an optional water electrolysis module 1600, and a CO-consuming process 640, in accordance with one embodiment of the disclosure. In embodiments not including an optional CO₂ concentration module 670 between the industrial process 610 and the removal module 620, the CO₂-comprising gas 612 from the industrial process 610 is passed to the removal module 620. In embodiments including an optional CO₂ concentration module 670 between the industrial process 610 and the removal module 620, the CO₂-comprising gas 614 from the industrial process 610 is passed to an optional CO₂ concentration module 670 to increase the concentration of the CO₂ in the gas stream 614 and remove one or more constituent 674. The gas stream having increased CO₂ concentration 672 from optional CO₂ concentration module 670 is passed to removal module 620, to remove and/or convert one or more constituents 628. In embodiments not including a CO₂ concentration module 670 between the removal module 620 and the CO₂ to CO conversion system 630, the treated stream 622 is passed from removal module 620 to CO₂ to CO conversion system 630. In embodiments including a CO₂ concentration module 670 between the removal module 620 and the CO₂ to CO conversion system 630, the treated stream 624 is then passed to an optional CO₂ concentration module 670 to increase the concentration of the CO₂ in the treated stream 624 and remove one or more constituents 674. The resulting CO₂ enriched stream 672 is passed from optional CO₂ concentration module 670 to CO₂ to CO conversion system 630 for conversion of at least a portion of CO₂ enriched stream 672.

Depending upon the CO₂ to CO conversion system 630 selected, O₂ may be generated, and if so, at least a portion of a O₂-rich gas stream 636 may be passed from the CO₂ to CO conversion system 630 to industrial process 610. At least a portion of CO-rich gas stream 632 may be passed to a further removal module 690 to remove and/or convert one or more constituents 698. At least a portion of the treated gas stream 634 may be passed to an optional O₂ separation module 660 to separate at least a portion of O₂ from treated gas stream 634. At least a portion of the O₂-enriched gas stream 664 may be passed from the optional O₂ separation module 660 to the industrial process 610. At least a portion of the O₂-depleted gas stream 662 may be passed from the optional O₂ separation module 660 to the further removal module 690 to remove and/or convert one or more constituents 698.

At least a portion of the gas stream 692 may be passed from the further removal module 690 to an optional pressure module 680. The pressurized gas stream 682 from the optional pressure module 680 is passed to CO-consuming process 640. The gas stream 692 may be blended with a H₂-rich gas stream 1602 prior to being introduced to the CO-consuming process 640. A water substrate 1606 may be passed a water electrolysis module 1600 to generate H₂-rich gas stream 1602 discussed above, and/or H₂-rich gas stream 1604 which may be passed from water electrolysis module 1600 directly to the CO-consuming process 640 via a conduit 1604. In some embodiments, O₂ produced by the water electrolysis module 1600 may be passed in O₂ stream 1608 to the industrial process 610.

The CO-consuming process 640 of FIG. 6 may produce product 646 and a post CO-producing process gaseous substrate 642 and 644. The CO-consuming process may be a gas fermentation process and may occur in an inoculator and/or one or more bioreactors. In embodiments wherein the CO-consuming process 640 is a gas fermentation process, a culture may be fermented to produce one or more fermentation products such as post CO-consuming process product 646 and a post-fermentation gaseous substrate, such as the post-CO-consuming process gaseous substrate 642 or 644. The post-CO-consuming process gaseous substrate 644 is passed to an optional CO₂ concentration module 670 to increase the concentration of the CO₂ in the gas stream 644 and remove one or more constituent 674. Resulting stream 672 is passed from optional CO₂ concentration module 670 to removal module 650 to remove and/or convert one or more constituent 658. The treated gas stream 652 is then passed to CO₂ to CO conversion system 630 for conversion of at least a portion of the gas stream. In particular embodiments, the post-CO-consuming process gaseous substrate 642 or 642/672 is passed to the same one or more removal modules 620 that receives the CO₂-comprising gas 612 or 672 from the industrial process 610. In various embodiments, the post-CO-consuming process gaseous substrate 642 or 642/672 may be passed to the one or more removal modules 620 that receives the CO₂-comprising gas 612 or 672 from the industrial process 610 and the treated gas stream 652 from the one or more removal modules 650.

In various embodiments, the disclosure provides an integrated process comprising electrolysis of water to provide at least hydrogen and optionally oxygen, wherein the power supplied for the water electrolysis process is derived, at least in part, from a renewable energy source.

Although the substrate is typically gaseous, the substrate may also be provided in alternative forms. For example, the substrate may be dissolved in a liquid saturated with a CO-comprising gas using a microbubble dispersion generator. By way of further example, the substrate may be adsorbed onto a solid support.

The C1-fixing microorganism in a bioreactor is typically a carboxydotrophic bacterium. In particular embodiments, the carboxydotrophic bacterium is selected from the group comprising Moorella, Clostridium, Ruminococcus, Acetobacterium, Eubacterium, Butyribacterium, Oxobacter, Methanosarcina, Methanosarcina, and Desulfotomaculum. In various embodiments, the carboxydotrophic bacterium is Clostridium autoethanogenum.

All references, including publications, patent applications, and patents, cited herein are hereby incorporated by reference to the same extent as if each reference were individually and specifically indicated to be incorporated by reference and were set forth in its entirety herein. The reference to any prior art in this specification is not, and should not be taken as, an acknowledgement that that prior art forms part of the common general knowledge in the field of endeavor in any country.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. The use of the alternative (i.e., “or”) should be understood to mean either one, both, or any combination thereof of the alternatives. As used herein, the term “about” means ±20% of the indicated range, value, or structure, unless otherwise indicated.

Recitation of ranges of values herein are merely intended to serve as a shorthand method of referring individually to each separate value falling within the range, unless otherwise indicated herein, and each separate value is incorporated into the specification as if it were individually recited herein. For example, unless otherwise indicated, any concentration range, percentage range, ratio range, integer range, size range, or thickness range is to be understood to include the value of any integer within the recited range and, when appropriate, fractions thereof (such as one tenth and one hundredth of an integer). Unless otherwise indicated, ratios are molar ratios, and percentages are on a weight basis.

All methods described herein can be performed in any suitable order unless otherwise indicated herein or otherwise clearly contradicted by context. The use of any and all examples, or exemplary language (i.e., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Embodiments of this disclosure are described herein. Variations of those embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

1. A process for improving carbon conversion efficiency comprising:
a. passing a CO₂-containing gaseous substrate from an industrial process, a synthesis gas process, or a combination thereof, to at least one removal module for removal of at least one constituent from the CO₂-containing gaseous substrate, to produce a treated gas stream, comprising at least a portion of CO₂;
b. passing the treated gas stream to a CO₂ to CO conversion system for conversion of at least a portion of the CO₂ to produce a first CO-enriched stream, wherein the CO₂ to CO conversion system is selected from reverse water gas shift reaction system, thermo-catalytic conversion system, electro-catalytic conversion system, partial combustion system, or plasma conversion system;
c. passing at least a portion of the first CO-enriched stream to a bioreactor comprising a culture of at least one C1-fixing microorganism; and
d. fermenting the culture to produce one or more fermentation products and a post-fermentation gaseous substrate comprising CO₂ and H₂;
e. passing at least a portion of the post-fermentation gaseous substrate comprising CO₂ and H₂ to at least one removal module for removal of at least one constituent from the post-fermentation gaseous substrate to produce a treated gas stream; and
f. recycling at least a portion of the treated stream to the CO₂ to CO conversion system.

2. The process of claim 1 wherein the CO₂ to CO conversion system is a reverse water gas shift reaction system and the process further comprising generating a H₂-rich stream using a water electrolyzer and passing and least a portion of the H₂-rich stream to the reverse water gas shift reaction system or to a location upstream of the reverse water gas shift reaction system.

3. The process of claim 1 further comprising passing at least a portion of the post-fermentation gaseous substrate comprising CO₂ and H₂ to at least one removal module for removal of at least one constituent from the post-fermentation gaseous substrate to produce a treated gas stream; and recycling at least a portion of the treated stream to the CO₂ to CO conversion system.

4. The process of claim 1, wherein the industrial process is selected from fermentation, carbohydrate fermentation, sugar fermentation, cellulosic fermentation, gas fermentation, cement making, pulp and paper making, steel making, oil refining, petrochemical production, coke production, anaerobic digestion, aerobic digestion, natural gas extraction, oil extraction, geological reservoirs, metallurgical processes, refinement of aluminium, copper and or ferroalloys, for production of aluminium, copper, and or ferroalloys, direct air capture, or any combination thereof; or
the synthesis gas process is selected from gasification of coal, gasification of refinery residues, gasification of petroleum coke, gasification of biomass, gasification of lignocellulosic material, gasification of waste wood, gasification of black liquor, gasification of municipal solid waste, gasification of municipal liquid waste, gasification of industrial solid waste, gasification of industrial liquid waste, gasification of refuse derived fuel, gasification of sewerage, gasification of sewerage sludge, gasification of sludge from wastewater treatment, gasification of biogas, reforming of landfill gas, reforming of biogas, reforming of methane, naphtha reforming, partial oxidation, or any combination thereof.

5. The process of claim 1, further comprising generating a H₂-rich stream using a water electrolyzer and
a. blending at least a portion of the H₂-rich stream with the CO-enriched stream prior to being passed to the bioreactor;
b. passing and least a portion of the H₂-rich stream to the bioreactor; or
c. both a) and b).

6. The process of claim 1, wherein the CO-enriched stream from the CO₂ to CO conversion system is passed to a removal module prior to being passed to the bioreactor.

7. The process of claim 1 wherein the at least one constituent removed from
a. the CO-enriched stream;
b. the CO₂-containing gas substrate; and or
c. the post-fermentation gaseous substrate;
is selected from sulfur-comprising compounds, aromatic compounds, alkynes, alkenes, alkanes, olefins, nitrogen-comprising compounds, oxygen, phosphorous-comprising compounds, particulate matter, solids, oxygen, halogenated compounds, silicon-comprising compounds, carbonyls, metals, alcohols, esters, ketones, peroxides, aldehydes, ethers, tars, and naphthalene.

8. The process of claim 7, wherein at least one constituent removed from the CO-enriched stream by the removal module comprises oxygen.

9. The process of claim 1, wherein at least one constituent removed and/or converted is a microbe inhibitor and/or a catalyst inhibitor.

10. The process of claim 1, wherein the at least one constituent removed is produced, introduced, and/or concentrated by the fermentation step.

11. The process of claim 1, wherein at least one constituent removed is produced, introduced, and/or concentrated by the CO₂ to CO conversion system.

12. The process of claim 1, wherein the C1-fixing microorganism is a carboxydotrophic bacterium.

13. The process according to claim 12, wherein the carboxydotrophic bacterium is selected from the group comprising Moorella, Clostridium, Ruminococcus, Acetobacterium, Eubacterium, Butyribacterium, Oxobacter, Methanosarcina, and Desulfotomaculum.

14. The process according to claim 13, wherein the carboxydotrophic bacterium is Clostridium autoethanogenum.

15. The process of claim 1, wherein the CO₂-containing gaseous substrate is passed to a carbon dioxide concentration module to enhance the level of carbon dioxide contained in (i) the CO₂-containing gaseous substrate prior to the CO₂-containing gaseous substrate being passed to the one or more removal module, (ii) the treated gas stream comprising at least a portion of carbon dioxide prior to the treated gas stream being passed to the water electrolyzer; and/or (iii) the post-fermentation gaseous substrate prior to the post-fermentation gaseous substrate being passed to the one or more removal modules, or the bioreactor.

16. The process of claim 1, further comprising passing the CO₂-containing gaseous substrate from the industrial process, the synthesis gas process, or the combination thereof to a pressure module to produce a pressurized CO₂-containing gas stream and then passing the pressurized CO₂-containing gas stream to the first removal module.

17. The process of claim 1, further comprising passing the CO-enriched stream to a pressure module to produce a pressurized CO-stream and passing the pressurized CO-stream to the bioreactor.

18. The process of claim 1, wherein at least one removal module is selected from hydrolysis module, acid gas removal module, deoxygenation module, catalytic hydrogenation module, particulate removal module, chloride removal module, tar removal module, or hydrogen cyanide polishing module.

19. The process of claim 1, wherein at least one of the fermentation products is selected from ethanol, butyrate, 2,3-butanediol, lactate, butene, butadiene, methyl ethyl ketone, ethylene, acetone, isopropanol, lipids, 3-hydroypropionate, terpenes, fatty acids, 2-butanol, 1,2-propanediol, or 1-propanol.

20. The process of claim 1, wherein at least one of the fermentation products is further converted to at least one component of diesel, jet fuel, and/or gasoline.

21. The process of claim 1, wherein at least one of the fermentation products comprises microbial biomass.

22. The process of claim 20, wherein at least a portion of the microbial biomass is processed to produce at least a portion of animal feed.

23. The process of claim 1, wherein the CO-enriched stream comprises at least a portion of oxygen, and at least a portion of the CO-enriched stream is passed to an oxygen separation module to separate at least a portion of oxygen from the carbon monoxide enriched stream.

24. A process for improving process economics of an integrated industrial fermentation system, the process comprising:
a. passing a feedstock comprising water to a water electrolyzer, wherein at least a portion of the water is converted to H₂ and O₂;
b. passing a CO₂-containing gaseous substrate to a reverse water gas shift process to generate a CO-enriched stream;
c. passing at least a portion of the CO-enriched stream from the reverse water gas shift process to a bioreactor containing a culture of at least one C1-fixing microorganism;
d. passing at least a portion of the H₂ to the reverse water gas shift process, to the bioreactor, or to both the reverse water gas shift process and the bioreactor;
e. fermenting the culture to produce one or more fermentation products and a post-fermentation gaseous substrate comprising CO₂ and H₂; and
f. passing at least a portion of the post-fermentation gaseous substrate back to the reverse water gas shift process.

25. The process of claim 24, wherein the amount of CO₂ in the post-fermentation gaseous substrate exiting the bioreactor is greater than an amount of unconverted CO₂ introduced to the bioreactor.

26. The process of claim 24, wherein the fermentation process performs the function of a CO₂ concentration module.
